(19) United States
(12) Patent Application Publication
(10) Pub. No.: US 2026/0120832 A1
(43) Pub. Date: Apr. 30, 2026

(54) SYSTEMS AND METHODS FOR GENERATING MEDICATION SUMMARIES

(71) Applicant: Oracle International Corporation, Redwood Shores, CA (US)

(72) Inventors: Yuwen Zhang, Kansas City, KS (US); Joseph James O’Malley, Mission, KS (US)

(73) Assignee: Oracle International Corporation, Redwood Shores, CA (US)

(21) Appl. No.: 19/368,762

(22) Filed: Oct. 24, 2025

Related U.S. Application Data
(60) Provisional application No. 63/712,370, filed on Oct. 25, 2024.

(51) Int. Cl. G16H 15/00; G16H 10/60; G16H 20/10; G16H 50/70

(52) U.S. Cl. CPC G16H 15/00; G16H 10/60; G16H 20/10; G16H 50/70

(57) ABSTRACT

A computer-implemented method includes receiving a request to provide a summary of patient-specific information regarding a medication for a particular patient and retrieving data relevant to the request from a plurality of sources, the retrieved data including at least structured and semi-structured content, harmonizing the retrieved data for at least data structure and semantic alignment to generate harmonized data, filtering the harmonized data to extract fields in the harmonized data relevant to the request, generating an input for a generative machine learning model, the input including a prompt generated based on the request, generating, by the generative machine learning model, a query result associated with the input, the query result including a subset of the filtered, harmonized data, formating the query result into an output, the output including the summary of patient-specific information regarding the medication, and providing the output to a client system.

### CROSS-REFERENCES TO RELATED APPLICATIONS

The present application is a non-provisional application of and claims the benefit and priority to U.S. Provisional Application No. 63/712,370, filed October 25, 2024, the entire contents of which is incorporated herein by reference for all purposes.

### BACKGROUND

Traditional healthcare systems involving computer-based assistants often rely on retrieving information from multiple data sources such as electronic health records and other data sources. Often these data sources code this data using custom coding schemes. As a result, retrieving and formatting retrieved information for standardized assistant user interfaces can be challenging. Traditional Electronic Health Record (EHR) systems can also have complex interfaces that may be difficult to navigate and cumbersome to operate. When the process for retrieving or recording necessary patient information is inefficient, it can disrupt the natural flow of the patient interaction.

Solutions addressing these changes and others would be desirable.

### SUMMARY

Techniques disclosed herein pertain to generative artificial intelligence (AI) systems, and, more specifically, to summary generation techniques using agentic AI systems.

In embodiments, a computer-implemented method includes receiving a request to provide a summary of patient-specific information regarding a medication for a particular patient and retrieving data relevant to the request from a plurality of sources. The retrieved data may include at least structured and semi-structured content. In embodiments, the method further includes harmonizing the retrieved data for at least data structure and semantic alignment to generate harmonized data, filtering the harmonized data to extract fields in the harmonized data relevant to the request, generating an input for a generative machine learning model, the input including a prompt generated based on the request. Further, the method includes generating, by the generative machine learning model, a query result associated with the input, the query result including a subset of the filtered, harmonized data. The method may further include formating the query result into an output, the output including the summary of patient-specific information regarding the medication, and providing the output to a client system.

In certain embodiments, the method includes transforming the retrieved data into a data format digestible by the generative machine learning model. In embodiments, the data includes a prescription history for the patient and guidance specific to the medication, and retrieving the data may include accessing the prescription history from an electronic health record (EHR) and obtaining the guidance specific to the medication from a medication database. The method may further provide the prompt to the generative machine learning model by instructing the generative machine learning model to analyze the prescription history and the guidance specific to the medication for contraindication for the medication in view of the prescription history. In certain embodiments, the data further includes additional information including at least one of the patient’s health history, the patient’s recent laboratory results, the patient’s vitals, and one or more clinical notes from the patient’s recent visits with a clinician making the request, and retrieving the data may further include accessing the additional information from the EHR. In embodiments, the data further includes insurance coverage information, and retrieving the data relevant to the summary may further include accessing the insurance coverage information from an insurance provider database. The method can further include providing the prompt to the machine learning generative model by instructing the machine learning generative model to review the insurance coverage information for the medication.

In embodiments, providing the output to the client system by presenting, at the client system, a medication summary report including at least one of a narrative summary section and a structured section. The narrative summary may include a first set of selections from the subset of the filtered, harmonized data arranged into natural language text, and the structured section may include a second set of selections from the subset of the filtered, harmonized data used to populate a plurality of preset windows within the medication summary report. In certain embodiments, at least one of the narrative summary and the structured section may include one of a Uniform Resource Locator (URL) and a hovering window to enable display of additional information regarding a specific portion of the summary of patient-specific information.

In embodiments, a system includes one or more processors and one or more computer-readable media storing instructions which, when executed by the one or more processors, can cause the system to perform operations including receiving a request to provide a summary of patient-specific information regarding a medication for a particular patient. In embodiments, the system retrieves data relevant to the request from a plurality of sources, the retrieved data including at least structured and semi-structured content. The system harmonizes the retrieved data for data structure and semantic alignment to generate harmonized data. The system can filter the harmonized data to extract fields in the harmonized data relevant to the request, and generate an input for a generative machine learning model, the input including a prompt generated based on the request. In embodiments, the system generates, by the generative machine learning model, a query result associated with the input, the query result including a subset of the filtered, harmonized data, format the query result into an output, the output including the summary of patient-specific information regarding the medication, and provide the output to a client system.

In certain embodiments, the system transforms the data into a data format digestible by the generative machine learning model. In embodiments, the data includes a prescription history for the patient and guidance specific to the medication, and retrieving the data includes accessing the prescription history from an electronic health record (EHR) and obtaining the guidance specific to the medication from a medication database. In embodiments, providing the prompt to the generative machine learning model includes instructing the generative machine learning model to analyze the prescription history and the guidance specific to the medication for contraindication for the medication in view of the prescription history. In certain embodiments, the data includes additional information including at least one of the patient’s health history, the patient’s recent laboratory results, the patient’s vitals, and one or more clinical notes from the patient’s recent visits with a clinician making the request, and retrieving the data includes accessing the additional information from the EHR. In certain embodiments, the data includes insurance coverage information, and retrieving the data relevant to the summary includes accessing the insurance coverage information from an insurance provider database. In embodiments, providing the prompt to the generative machine learning model includes instructing the generative machine learning model to review the insurance coverage information for the medication. In certain embodiments, providing the output to the client system includes presenting, at the client system, a medication summary report including at least one of a narrative summary section and a structured section. The narrative summary may include a first set of selections from the subset of the filtered, harmonized data arranged into natural language text, and the structured section may include a second set of selections from the subset of the filtered, harmonized data used to populate a plurality of preset windows within the medication summary report. In embodiments, at least one of the narrative summary and the structured section includes a Uniform Resource Locator (URL) or a hovering window to enable display of additional information regarding a specific portion of the summary of patient-specific information.

In embodiments, one or more non-transitory computer-readable media store instructions which, when executed by one or more processors, cause the one or more processors to perform operations including receiving a request to provide a summary of patient-specific information regarding a medication for a particular patient. In embodiments, operations further include retrieving data relevant to the request from a plurality of sources, wherein the retrieved data includes at least structured and semi-structured content. The operations further include harmonizing the retrieved data for data structure and semantic alignment to generate harmonized data, and filtering the harmonized data to extract fields in the harmonized data relevant to the request. The operations further include generating an input for a generative machine learning model, the input including a prompt generated based on the request, and generating, by the generative machine learning model, a query result associated with the input, the query result including a subset of the filtered, harmonized data. The operations further include formatting the query result into an output, the output including the summary of patient-specific information regarding the medication, and providing the output to a client system.

In certain embodiments, harmonizing the data includes transforming the data into a data format digestible by the generative machine learning model.

### BRIEF DESCRIPTION OF THE DRAWINGS

The present disclosure is described in conjunction with the appended figures, where like components are indicated with like reference numbers.

FIG. 1 is a block diagram illustrating an example computing environment incorporating agent-driven services, according to certain environments.

FIG. 2 is a block diagram illustrating an example of the function of portions of the cloud service provider platform of FIG. 1 in producing a medication summary, in accordance with embodiments.

FIG. 3 is an example response sent to the client device, in accordance with embodiments.

FIG. 4 is a flowchart illustrating an example process for generating a medication summary in accordance with a user-provided query, in accordance with embodiments.

FIG. 5 is a flowchart illustrating an alternative process for generating a medication summary in accordance with a user-provided query, in accordance with embodiments.

FIG. 6 is a flowchart illustrating a portion of the processes shown in FIGS. 4 and 5, in accordance with embodiments.

FIG. 7 a block diagram illustrating an example computing environment incorporating an agent-driven digital assistant system, in accordance with embodiments.

FIG. 8 is a block diagram illustrating one pattern for implementing a cloud infrastructure as a service system, according to at least one embodiment.

FIG. 9 is a block diagram illustrating another pattern for implementing a cloud infrastructure as a service system, according to at least one embodiment.

FIG. 10 is a block diagram illustrating another pattern for implementing a cloud infrastructure as a service system, according to at least one embodiment.

FIG. 11 is a block diagram illustrating another pattern for implementing a cloud infrastructure as a service system, according to at least one embodiment.

FIG. 12 is a block diagram illustrating an example computer system, according to at least one embodiment.

### DETAILED DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

### INTRODUCTION

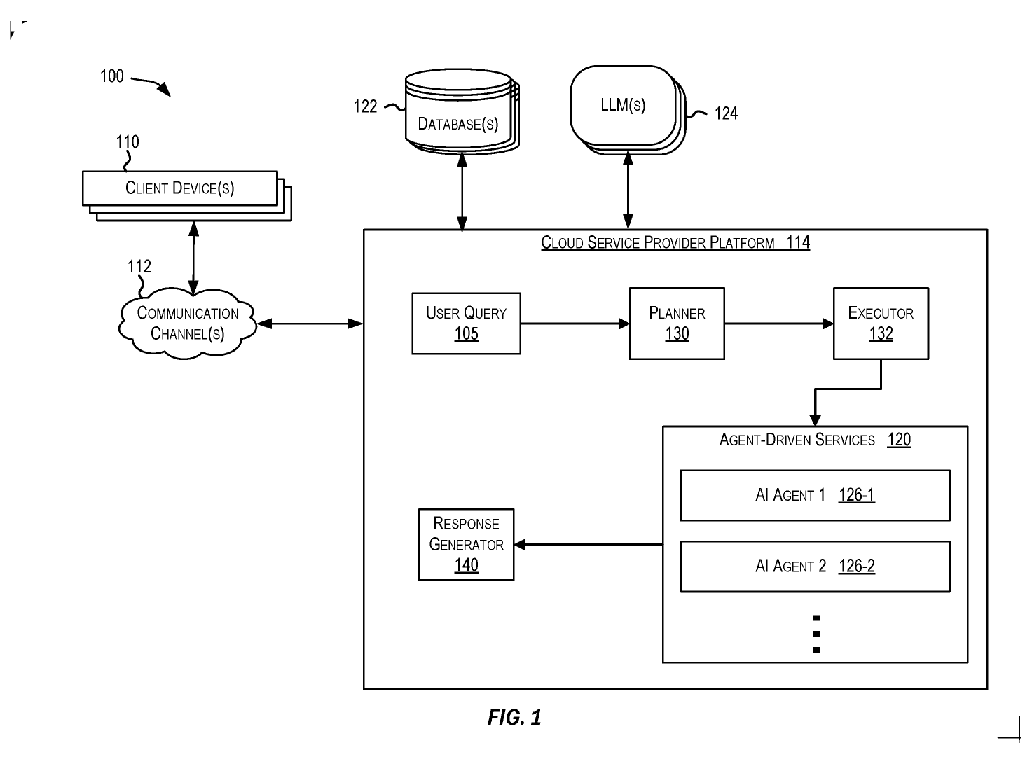

Healthcare providers often need to locate and review a variety of information regarding a patient prior to an encounter, but it can be difficult to locate, review, and discern the relevant information, even with the proliferation of electronically accessible patient electronic health record (EHR) systems. In many cases, healthcare providers employ intelligent tools such as agentic digital assistants to perform these tasks. These agentic digital assistants often utilize one or more generative machine learning models such as large language models (LLMs) to retrieve information related to an inquiry from multiple sources such as EHRs, knowledge bases, databases, and the like, process the information, and generate a response to the inquiry from the processed information. An example of such an approach is discussed in U.S. Pat. App. No. 18/624,472, filed April 2, 2024, which is incorporated herein by reference as if fully set forth herein. As shown in FIG. 1, agent-driven services 120 may include, for example, one or more artificial intelligence agents (hereinafter “AI agent 126”). In an embodiment, agent driven services 120 includes a plurality of AI agents, shown as AI Agent 1 (126-1), AI Agent 2 (126-2), and so on, as indicated by ellipsis. Each AI agent may be configured to specialize in a particular task, such as the modular summary generation disclosed herein. In examples, an AI Agent may call one or more of LLMs 124 to generate an execution plan (e.g., instructions), then execute the execution plan. In embodiments, an agent may itself include a model, such as an LLM, small language model (SLM), medium language model (MLM), a machine learning model, or others. Name Known conditions (e.g., for a specified time frame and a range of conditions relevant to the medication in the query) Stated reason for visit (RFV), if the query is generated prior to or before a patient encounter Chief complaint (CC), as relevant to the medication in the query Last Visit Date and Type (e.g., wellness, follow-up, acute) Current and New Allergies Since Previous Visit New Related Diagnostic Study Data and Lab Results Since Previous Visit New Related Procedures Related Social History Related Family History Current Intake Notes (if a patient encounter) The pulled information from EHR database 212 may be filtered for relevancy using, for example, a semantic knowledge graph, specifying known connections between different pieces of information. For example, if a specific patient has a history of diabetes, then data loading block 210 may only pull information related to diabetes medications. If the specific medication is known to have contraindications related to another condition with which the patient has previously been diagnosed, then such related information may also be used in the filtering process. Similarly, if the particular medication in medication query 207 affiliated with a different medication in the patient record (e.g., if a medication A in the medication query is chemically or functionally similar to a medication B, which was previously prescribed to the patient for a different condition), then such affiliate information may be extracted from EHR database 212 at data loading block 210. The filtering may further take into consideration the timing of the information, such as giving more weight to conditions and/or medications affecting a particular patient in the past six weeks over older conditions beyond six months in a sliding window approach.

While these agentic digital assistants have been useful in improving information retrieval and synthesis, utilizing these assistants in clinical settings presents challenges. For example, EHRs often encompass extensive and fragmented information, including personal information, patient histories, test results, physician notes, and medication records stored using different coding schemes, but processing this vast context efficiently poses a significant challenge for a variety of reasons such as information overload, model limitations, temporal context, and patient-specific context. In another example, EHR data is rarely presented in a unified format with both structured fields (e.g., lab results, medication lists) and unstructured text (e.g., physician notes, patient complaints), but processing these different formats poses data fusion and aggregation challenges, semantic alignment challenges, and inconsistencies across healthcare providers). In yet another example, LLMs and other generative machine learning models are often pre-trained on general concepts, but lack a deep understanding of clinical contexts, guidelines, textbooks, publications, ontologies, and medical reasoning, which often results in inaccuracies and can have severe consequences such as misdiagnosis and/or inappropriate treatments.

The techniques described herein overcome these challenges and others by providing techniques for generating clinical summaries, and more particularly, to medication history summaries.

A simplified example of an agentic AI approach to receiving user queries from client devices, extracting data from multiple databases, processing the user queries by managing multiple agent-driven services, then producing a result is illustrated in FIG. 1. FIG. 1 is a block diagram illustrating an example computing environment incorporating agent-driven services, according to certain environments. In examples, the agent-driven services may include one or more artificial intelligence resources acting as “agents,” each performing a defined set of tasks. For instance, one of the agents may implement the summary of patient-specific information, as described herein. In an embodiment, computing environment 100 includes one or more client devices 110 (hereinafter “client devices 110”), one or more communication channels 112 (hereinafter “communication channels 112”), a cloud services provider platform 114 (hereinafter “platform 114”) including agent-driven services 120 and connected with one or more databases 122 (hereinafter “databases 122”) and one or more large language models 124 (hereinafter “LLMs 124”).

While the present disclosure mentions the use of LLMs as an example mechanism for analyzing data and generating patient information summaries, it is noted that other generative artificial intelligence techniques, including other generative machine learning models may be used including. Examples of such techniques and models include, but are not limited to, Small Language Models (SLMs), Multi-modal Models, reasoning models and chain-of-thought architectures, transformer-based models, and the like.

Cloud services provider platform 114 receives user query 105 from one of client devices 110 via communication channels 112, and user query 105 is passed to a planner 130. In embodiments, planner 130 determines the appropriate course of action (e.g., selection of the appropriate AI agent with agent-driven services 120, timing and/or prioritization of tasks to be performed in response to the user query, etc.), then the action so determined is sent to executor 132.

In embodiments, at executor 132, a new execution plan may be generated or an existing plan selected out of a library of execution plans (not shown). An execution plan may include, for example, information regarding the course of action, timing, prioritization, etc. The execution plan is then performed by one or more of the AI agents within agent-driven services 120. The one or more of the AI agents performs the appropriate tasks, based on the information accessible at databases 122 and LLMs 124, to send the resulting output to a response generator 140. Response generator 140 generates then transmits a response to the client device that originated the user query 105.

### Medication Summary Generation System

In contrast with existing AI systems for generating summaries of specific documents or sets of data, the embodiments described herein provide innovative routing models for extracting and filtering data from data sources within EHR systems as well as external sources, while enabling efficient generation and updating of semantic objects (e.g., medication summaries) for consumption by medical professionals in clinical settings. In implementations, the embodiments disclosed herein may be implemented, for example, as one or more of the AI agents 126 of FIG. 1.

It is noted that, the term “healthcare provider” generally refers to healthcare practitioners and professionals including, and not limited to: physicians (e.g., general practitioners, specialists, surgeons, etc.); nurse professionals (e.g., nurse practitioners, physician assistants, nursing staff, registered nurses, licensed practical nurses, etc.); and other professionals (e.g., pharmacists, therapists, technicians, technologists, pathologists, dietitians, nutritionists, emergency medical technicians, psychiatrists, psychologists, counselors, dentists, orthodontists, hygienists, etc.).

As a technical challenge, extracting information from a variety of medical record sources with different coding schemes may be difficult for a computing system. For example, many EHR systems use proprietary coding systems for storing patient information such that, while the stored medical information may be standardized within a specific EHR, many different coding schemes are used amongst different EHR systems.

Currently available EHR systems are generally focused on data intake (i.e., ensuring data are entered into the EHR in uniform formats that are readily retrievable), creating reminders based on the EHR information, and creating summary reports using information stored within the EHR system. For example, in traditional approaches of computing a summary report in an automated fashion, the generation process is performed in an “end-to-end” fashion, i.e., by retrieving all of the required data from a semantic index (e.g., EHR) and performing a “one-shot” call to a resource such as a large language model. Such an approach is computationally intensive and time consuming.

Further, in assessing a patient health as a whole, the necessary information may lie outside of the EHR system itself. For instance, regulatory, insurance, pharmaceutical, and other information may be required in determining whether a particular medication is suitable for prescription to a patient, given the patient’s known health history. Such a determination requires a thorough consideration of the patient’s known conditions and allergies, past illnesses, current and past prescriptions, and other information that may exist within an EHR system.

Additionally, data that exist outside of the EHR, such as indications and any counter-indications for the particular medication, alone or in combination with other medications, regulatory considerations (e.g., as related to controlled substances), insurance coverage, generic alternatives for the medication, efficacy data, publications related to the medication, and others.

Moreover, beyond simply being able to obtain data, it would be highly desirable to be able to identify trends in the data over time and as correlated between different pieces of information, such as changes in health condition (as observed by a healthcare provider, self-reported by the patient, indicated by lab results, etc.) with changes in medication or other patient conditions. Particularly for patients with complex health conditions, a medication change has numerous implications that must be considered in by the healthcare provider.

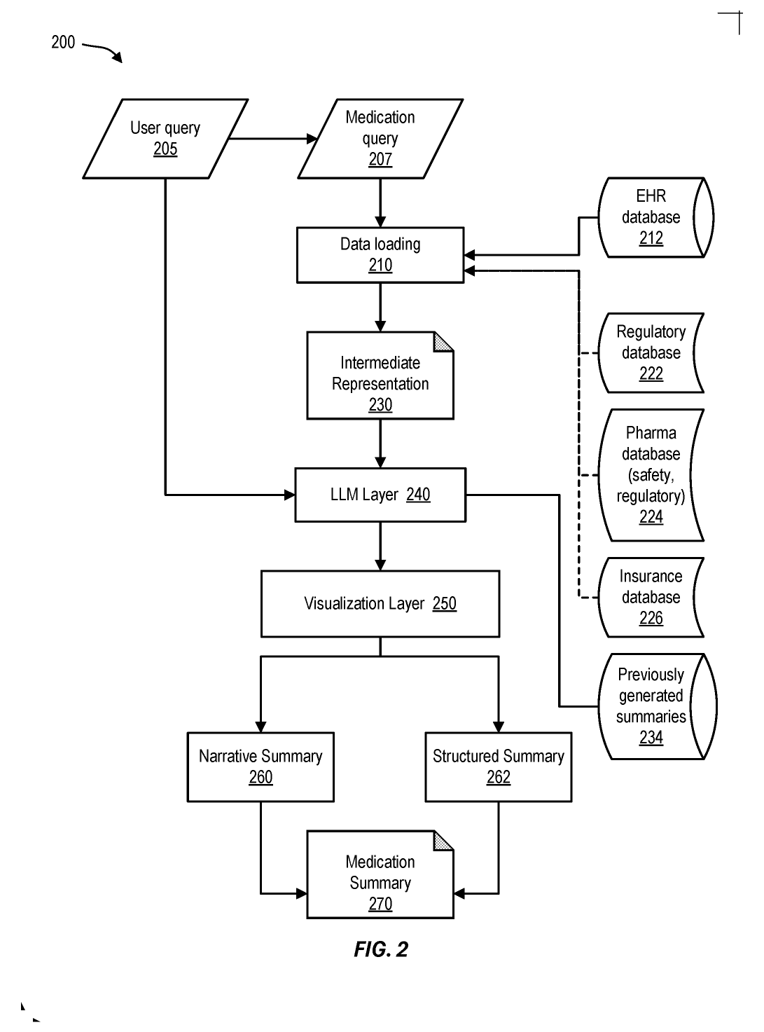

FIG. 2 is a block diagram illustrating an example of the function of portions of the cloud service provider platform of FIG. 1, in accordance with embodiments. The initial patient summary may be suitable, for example, for use as a medication summary to be quickly reviewed by a healthcare provider prior to, during, and/or in preparation for a patient encounter.

As shown in FIG. 2, a system 200 begins with receipt of a user query 205 (such as user query 105 of FIG. 1). In particular, user query 205 includes known terminology or keyword(s) related to a medication such as, and not limited to, “medication,” “prescription,” “remedy,” drug,” name of a known medication, and others. In embodiments, recognition by cloud service provider platform 114 that user query 205 (e.g., user query 105 in FIG. 1) includes medication-related terminology may trigger planer 130 and executor 132 to initiate a medication query 207, which is fed into a data loading block 210. Medication query 207, and subsequent processing blocks described below, may be implemented as one of AI agents within agent-driven services 120, for example.

Data loading block 210 functions to pull, filter, and process data from one or more EHR databases 212 (e.g., database(s) 122 of FIG. 1), as an EHR system generally includes many semantic objects in structured (e.g., intermediate representations, data strings with standardized headers and metadata, etc.) and semi-structured formats (e.g., JSON/XML forms and clinical documents, templated notes, HL7 v2 messages, templated portable document format (PDF) documents, data with Clinical Document Architecture (CDA), Continuity of Care Document (CCD), and/or Digital Imaging and Communications in Medicine (DICOM) standard headers to name a few examples) and many of which may not be relevant to the medication or the specific patient.

For instance, data loading block 210 retrieves patient-specific information from the EHR database including, and not limited to:

- Name

- Age

- Gender

- Most recent vitals (height, weight, heart rate, blood pressure, etc.)

- Current and past prescriptions and medications (e.g., for a specified time frame)

- Known conditions (e.g., for a specified time frame and a range of conditions relevant to the medication ion the query)

- Stated reason for visit (RFV), if the query is generated prior to or before a patient encounter

- Chief complaint (CC), as relevant to the medication in the query

- Last Visit Date and Type (e.g., wellness, follow-up, acute)

- Current and New Allergies Since Previous Visit

- New Related Diagnostic Study Data and Lab Results Since Previous Visit

- New Related Procedures

- Related Social History

- Related Family History

- Current Intake Notes (if a patient encounter)

- Previous recommendations (e.g., all past and home medications related to a given medication or class of medications, historical prescriptions in the same category (e.g. statins), including dosage changes, medications tried and failed in lieu of this medication, medication change due to adverse reactions, insurance reasons or prohibitive cost reasons)

- Future related appointments

- Current Related Messages from Message Center (e.g., an inquiry regarding a prescription refill or a new medication)

The pulled information from EHR database 212 may be pulled for relevancy using, for example, a semantic knowledge graph, specifying known connections between different pieces of information. For example, if a specific patient has a history of diabetes, then data loading block 210 may only pull information related to diabetes medications. If the specific medication is known to have contradictions related to another condition which he patient has previously been diagnosed, then such related information may be used in the filtering process. Similarly, if the particular medication in medication query 207 affiliated with a different medication in the patient record (e.g., ia a medication A in the medication query is chemically or functionally similar to a medication B, which was previously prescribed to the patient for a different condition), then such affiliate information may be extracted from EHR database 212 at data loading block 210. The filtering may further take into consideration the timing of the information, such as giving more weight ro conditions and/or medications affecting a particular patient in the past six weeks over older conditions beyond six weeks in a sliding windows approach.

Additionally, the data pulled by data loading block 210 may be processed for uniformity. For instance, semantic objects pulled from EHR database may include both structured data (e.g., numerical data, information stored in standardized formats such using as Fast Healthcare Interoperability Resources (FHIR) standards or an internal standard format) as well as unstructured data (e.g., scanned, handwritten documents, freeform note entries, recorded voice memos). In this case, the extracted data may be processed, for example, to add data identification information, supplemented with metadata, digitized (e.g., using optical character recognition, natural language processing, and/or image processing methods), and other techniques to put the retrieved data into a format suitable for subsequent processing by agent-driven services.

Optionally, as indicated by dashed arrows, data loading block 210 may also be configured to connect with and extract data from data sources other than the EHR database. For example, data loading block 210 may look for information relevant to medication query 207 at internal and external data stores such as, and not limited to, a regulatory database 222 (e.g., containing public and/or private information regarding the regulatory guidance and approval status of specific medications, any rules and regulations related to prescription of the relevant medications), a pharmaceutical database 224 (e.g., public and/or private data related to medication efficacy, indications and contraindications, availability of generic versions, research studies, and clinical data), and an insurance database 226 (e.g., including insurance coverage information, pricing, reimbursement codes, etc.). Data from such disparate sources are also likely to require filtering and formatting, in order to homogenize the data for subsequent processing steps.

In embodiments, data loading block 210 filters the extracted data and semantic objects from the various databases for only the information potentially relevant to the medication query 207, then processes the resulting dataset into intermediate representation (IR) 230, with a standard syntax suitable for submission to a Large Language Model (LLM) layer 240. For example, IR 230 has been transformed to have a structured, computer-interpretable encoding format, such as used in commonly used in natural language processing. In other words, data loading block further processes the extracted data to harmonize the data with structured and semi-structured formats that may have been obtained from a plurality of data sources. Thus, data loading block prepares the extracted data for ready processing in subsequent handling steps as described below.

LLM layer 240 (e.g., one of LLMs 124 in FIG. 1) is used to generate a collection of phrases and sentences that may be used as a part of a response created for the originator of the user query. LLM layer 240 may also include a repository of commonly used prompts and context statements suitable for medication-related queries, such as, “As a clinician, I want a medication summary focused on a single patient condition, presented in a list format, with the most relevant medications at the top of the list.”

The collection of phrases and sentences are presented to a visualization layer 250, which generates the contents of a narrative summary 260 and a structured summary 262. For example, LLM layer 240 may process IR 230 through natural language processing methods to generate suitable components of narrative summary 260 based on the extracted information. In examples, narrative summary provides an informative summary of patient information, particularly highlighting the relevant medications and conditions as gleaned from the medication query 207 as well as data pulled by the data loading block 210 from one or more data sources.

In certain embodiments, rather than presenting all relevant information at once on the screen, certain keywords and/or phrases may be embedded with uniform resource locator (URL) links to information in intermediate representation 230 and/or LLM layer 240. For example, clicking on or hovering a pointer over a keyword or phrase with an embedded URL may open a hovering window over the existing screen, with the hovering window containing additional information about the keyword, showing an image of a handwritten document or medical image, providing a visual representation of numerical data trends, a webpage with source information, or others. In this way, the amount of data presented to the user on a screen may be limited, even with a thorough narrative summary, while allowing the option of retrieving more detailed information via the URL links.

Narrative summary 260 may be provided as a standalone report and/or combined with other facts organized by LLM layer 240 into a structured format. For instance, structured data as extracted and processed at data loading block 210 then further processed by LLM layer 240 may be used by visualization layer 250 to populate a structured summary 262. The structured data may include, for example, facts and numerical information as related to known medications, lab results, and other structured information that may be directly inserted into a form, added as a universal resource locator (URL) link, etc. Visualization layer 250 crafts a medication summary 270, using the narrative summary 260 and/or structured summary 262, for presentation to the user in response to the original query. Visualization layer layer 250 may also configure the formatting of any hovering window that pops up when a user clicks on or hovers over a URL in the medication summary.

In embodiments, narrative summary 260, structured summary 262, and/or medication summary 270 may be stored in memory at the cloud service provider platform (e.g., cloud service provider platform 114 of FIG. 1) until specifically requested by the user. Particularly if the medical query 207 was automatically triggered by the cloud service provider platform upon detection of medication-related terminology in the original user query, then there is a possibility that user query 205 may have triggered functionalities of the cloud service provider platform, such as other agentic AI processes. In such a case, planner 130 of cloud service provider platform may provide prioritization instructions to the various agent-driven services to present any outputs from the AI agents in an order appropriate for a specific user case scenario.

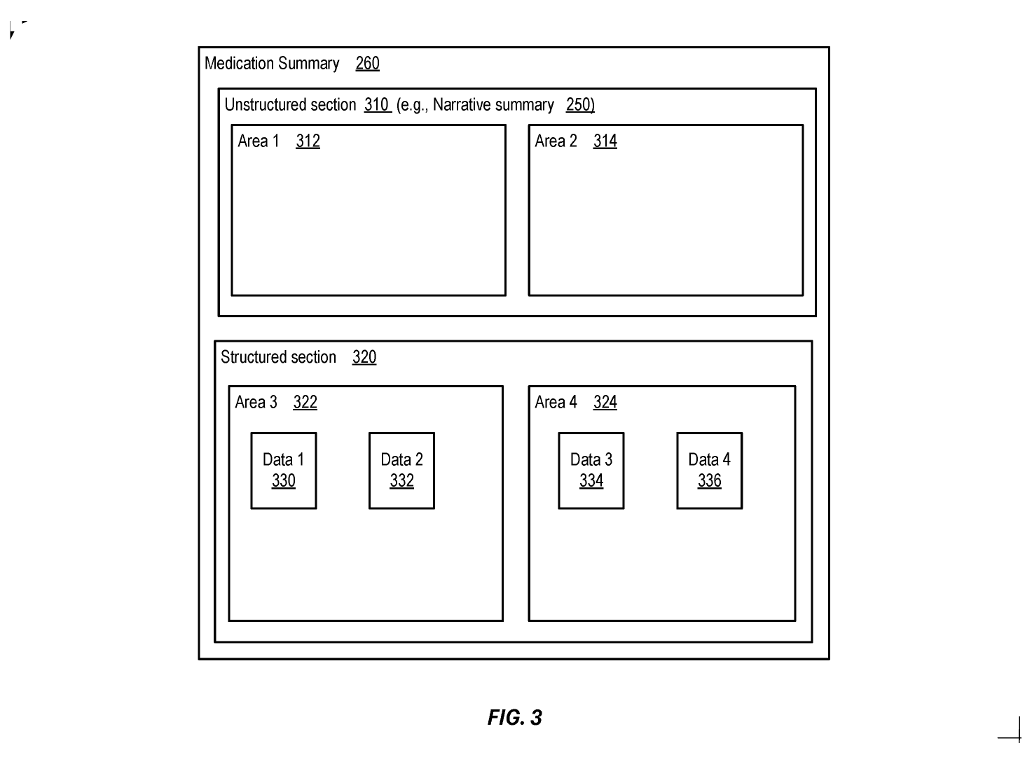

An example presentation format of medication summary 270 is shown in FIG. 3. As shown in FIG. 3, which shows an example response sent to the client device, in accordance with embodiments, medication summary 270 includes an unstructured section 310 (including, as an example, narrative summary 260 of FIG. 2). Unstructured section 310 may include one or more areas (shown as area 1 (312) and area 2 (314)), into which narrative related to specific topics may be inserted. For instance, area 1 (312) may be used to present a general summary of a medication that is the subject of medication query 207, while area 2 (314) may be reserved for a summary of recent prescription history, known allergies, and other information that may be drawn from various sections of the database(s) from which medication-related information have been extracted.

The information presented in unstructured section 310 may be an extract from narrative summary 260 or independently populated using extracted IR 230. In embodiments, unstructured section 310 may include a generated summary of the relevant information in natural language format, bullet points of key information, extracts from notes from previous patient encounters, electronic scans of historical notes, and other long-form information.

Medication summary 270 may additionally include a structured section 320. In the illustrative example, structured section 320 may include one or more areas (shown as area 3 (322) and area 4 (324)), in which structured data such as lab test results and lists of known allergies and conditions may be presented. Area 3 (322) and area 4 (324) may include structured data extracted from IR 230 used to populate an predetermined template. For example, area 3 (322) may include data 1 (330) presented as a list of past patient conditions next to data 2 (332), including a list of known medications relevant to the medication query, or a list of indications and contraindications for the medication. Similarly, area 4 (324) may include data 3 (334), with a graph visually showing the evaluated numbers from previous lab test results, along with data 4 (336) with a list of links containing URLs linking to external or internal repository of information related to explanation of the relevant terminology. Such visual representation of structured data may assist the healthcare provider in identifying trends as related to the rest of the patient medical history. Optionally, the information presented in the structured section may have been further processed, for example, to emphasize the most recently entered information, such as the list of other conditions diagnosed over the past six months. In certain embodiments, medication summary 270 may include a search field or a user interface “button” to allow the healthcare provider to regenerate the medication summary based on any newly added information and the previously extracted IR 230. If necessary, additional information may be pulled from one or more databases to be added to IR 230. In certain embodiments, the healthcare provider may be presented with an option to save the current summary report to a file folder in memory, prior to generating an updated summary report. For instance, as a particular patient may present with multiple medical conditions, additional and/or updated summary reports may be necessary for a complete assessment of the patient history. As another example, certain portions of the unstructured section may be enhanced with structured information, such as URL links to access external documents (e.g., images of handwritten notes).

In this way, medication summary 270 enables healthcare providers to obtain a trove of information regarding a medication as related to a specific patient’s health history in a compact format, suitable for display on a small screen such as a tablet, as well as links to additional information of so desired. In embodiments, one or more portions of medication summary 270 may be reserved to allow the healthcare provider to enter additional notes.

In certain embodiments, medication summary 270 may include a search field or a user interface "button" to allow the healthcare provider to regenerate the medication summary based on any newly added information and the previously extracted IR 230. If necessary, additional information may be pulled from one or more databases to be added to IR 230. In certain embodiments, the healthcare provider may be presented with an option to save the current summary report to a file folder in memory, prior to generating an updated summary report. For instance, as a particular patient may be present with multiple medical conditions, additional and/or updated summary reports may be necessary for a complete assessment of the patient history. As another example, certain portions of the unstructured section may be enhanced with structured information, such as URL links to access external documents (e.g., images of handwritten notes)

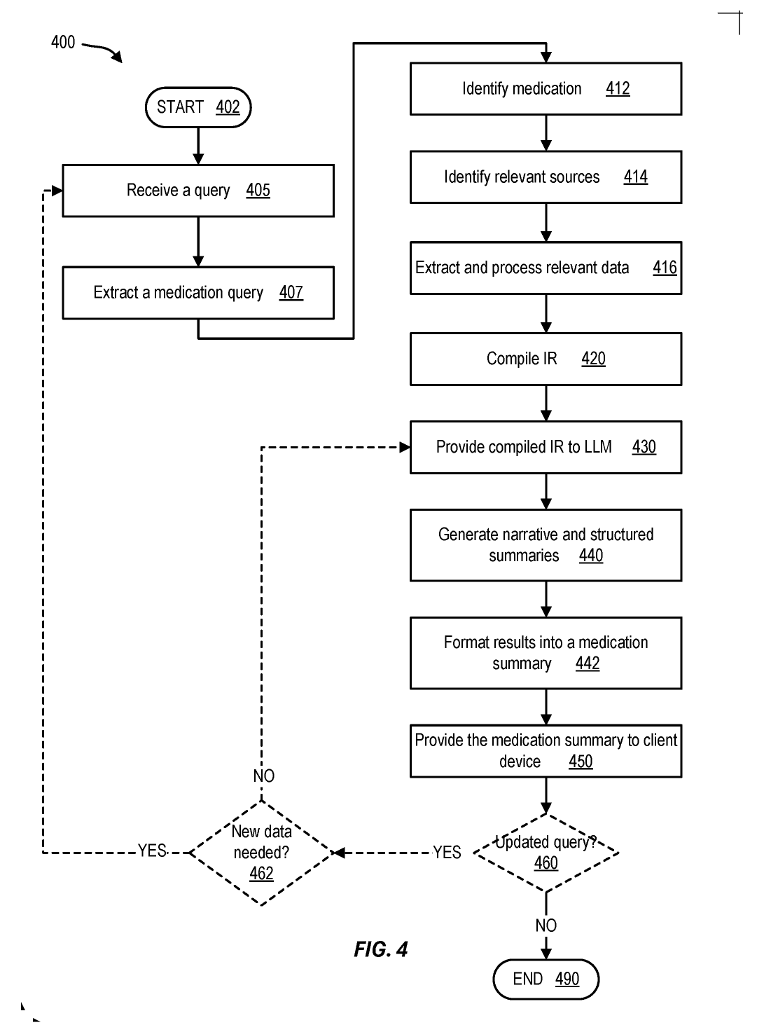

An example process suitable for use in generating medication summaries as described above is illustrated in FIG. 4, showing a flowchart illustrating a process for generating a response in accordance with a user-provided query, in accordance with embodiments. A process 400 as shown in FIG. 4 may be performed in a computing environment (e.g., by cloud service provider platform 114 of FIG. 1).

Process 400 is initiated in a start step 402, then proceeds to receive a query in 405. The query may have been received from a client device (e.g., client device 110) or automatically generated by a component or service within or outside of cloud service provider platform 114. In the example process 400, a medication query is extracted from the received query in 407. As discussed above, the medication query may be automatically triggered by the cloud service provider platform by identification of terminology related to medication. Alternatively, the query may specifically relate to a medication (e.g., “what are the contraindications for medication A if being used to treat condition X?”).

The specific medication in the medication query is identified in block 412. In block 414, the process proceeds to identify the relevant sources of data from which the query-related data should be pulled, such as a part of the functionality of data loading block 210 of FIG. 2. For example, if a known controlled substance is the subject of the medication query, then relevant sources may include a regulatory database including prescription guidance, portions of an EHR system including past patient prescription records, and a repository of pharmaceutical information regarding diagnosed conditions that may be treated by the specified medication. If the medication query relates to an expensive and/or experimental drug, then the relevant sources may further include a scientific publication archive with clinical studies and an informational database provided by the specific type of insurance used by the patient, in order to determine whether the treatment involving the drug is covered by the patient’s insurance.

In another aspect of the functionality of data loading block 210 of FIG. 2, process 400 proceeds to extract and process the relevant data from the data sources so identified. The extraction may take into consideration a variety of patient and medication data, such as a category of the patient encounter as selected from preset categories such as, and not limited to, an acute/urgent care visit, an annual/wellness visit, a follow-up visit as related to a previous encounter, and a generic/uncategorized visit. As discussed above, processing may include, for example, harmonizing the retrieved data with the necessary formatting, identification data, metadata, etc. The processed data is then compiled in block 420 as a set of intermediate representation of the retrieved semantic objects such that the IR is readily digestible by an LLM.

The IRs as compiled are then provided to a generative resource, such as a trained LLM, in block 430, which in turn generates narrative and structured summaries in block 440. The results of block 440 may then be formatted into a medication summary (e.g., medication summary 270 of FIG. 2) in block 442, and the medication summary may be provided to the client device or the originator of the query in block 450. In certain embodiments, rather than providing the medication summary to a user, the IR, narrative summary, structured summary, and/or medication report may be stored in memory at the cloud service provider platform for future use.

Multiple approaches are contemplated for the generation of the narrative and structured summaries and are considered to be a part of the present disclosure. For example, zero-shot prompting, one-shot prompting, chain-of-thought prompting may be considered for all or different aspects of the summary semantic object generation.

Optionally, a decision may be made in a determination 460 whether an updated or new query related to medication has been received, such as entered at a client device user interface, based on a search performed by the healthcare provider reading the summary report, clickthroughs to URLs provided as part of the summary report, and other information. For instance, in a patient encounter, whereas the recorded reason for visit was for one complaint, thus prompting a query regarding a first medication, the healthcare provider may discover the patient may have a different complaint that is more severe than the original RFV, requiring information regarding a second medication.

If an updated query has been received, then a determination 462 is made whether new or additional data is required to respond to the updated query. If determination 462 results in YES, new data is needed (e.g., if a new condition or medication has been identified), then process 400 returns to block 405 to process the updated query anew. If determination 462 results in NO, new data is not needed and the new query may be addressed using the extracted initial IR, the process 400 returns block 430 to process the relevant data anew in view of the updated query, thus enabling savings in computational time and resources. If no updated query has been received, then process 400 is terminated at end step 490.

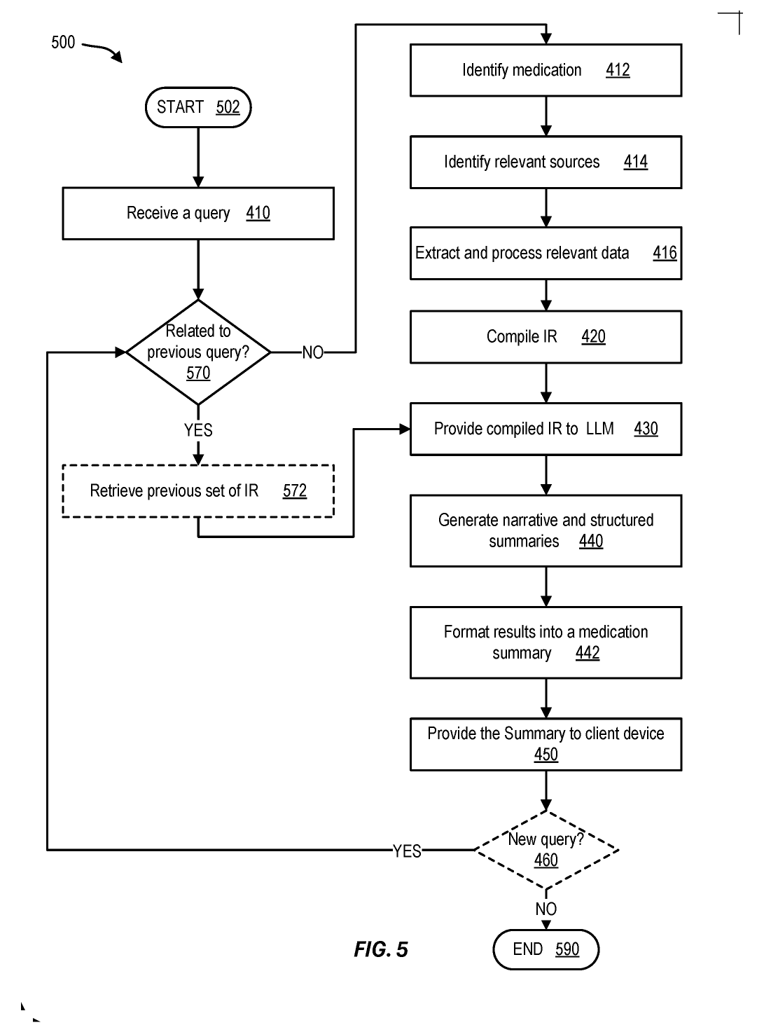

FIG. 5 illustrates an alternative process in which every query is evaluated whether it is related a previously presented query. As shown in FIG. 5, which is a flowchart illustrating an alternative process for generating a medication summary in accordance with a user-provided query, in accordance with embodiments, a process 500 shares several of the processing blocks as shown in FIG. 4. However, upon initiating process 500 at a start step 502 and a query is received in block 410, a determination 570 is made whether the received query is related to a previously presented query. If determination 570 results in NO, that the received query is unrelated to previously submitted queries, then process 500 proceeds to block 412 and follows the processing steps as shown in FIG. 4.

If determination 570 yields YES, the presently received query is related to a previous query, then process 500 optionally proceeds to retrieve a previously extracted set of IR (e.g., from cached records at the client device or within the cloud services platform) in block 572 then to soft filtering block 420. Alternatively, for example if the previously extracted IR is still active at the client services platform, then process 500 may immediately proceed to perform soft filtering anew at block 420.

The techniques described in FIGS. 4 and 5 allows efficient production of multiple medication summaries. For example, information for a given combination of medication and patient may be cached and shared as at least a portion of the extracted IR in addressing a new or updated query. The shared information may include, for example, conditions or medications for the patient, lab tests or vitals relevant to different conditions, family history, and previous summarized A&P from previous patient encounters. In embodiments, when a summary generation is requested, then the pre-computed modules (i.e., extracted and/or filtered IRs) may be loaded to maximize reuse of semantic objects that have already been processed, then the patient summaries may be generated using minimal computational resources (e.g., LLM tokens).

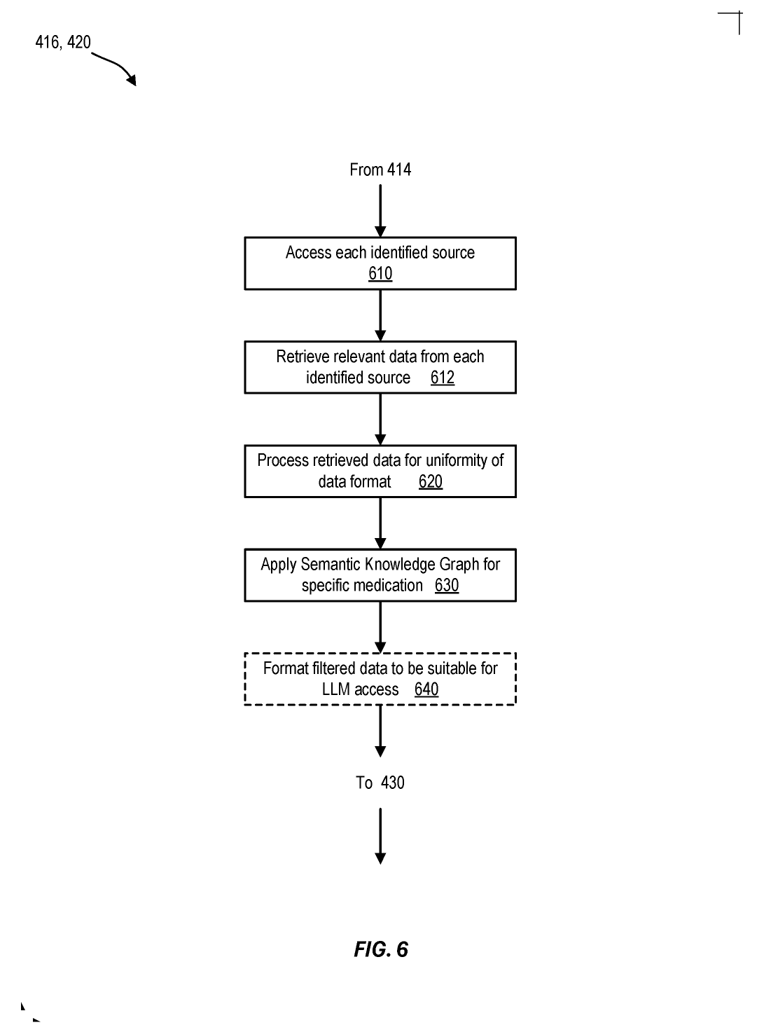

FIG. 6 shows additional details of the processing performed at blocks 416 and 420 of FIGS. 4 and 5, in an embodiment. As shown in FIG. 6, which shows a flowchart illustrating a portion of the processes shown in FIGS. 4 and 5, in accordance with embodiments. After relevant sources are identified in block 414, each identified source is accessed in block 610, and relevant data retrieved in block 612. Then the retrieved relevant data are processed for uniformity of data format, such as in an industry standard format, a standardized format for a given EHR, to name a few examples. The harmonized data may be further filtered using, for instance, a semantic knowledge graph as discussed above for specific medication as applied to a particular patient. Further, the filtered data may optionally be further formatted in a manner suitable for processing with one or more LLMs in generating the narrative summary in block 620 as well as for use in the structured summary section in block 630, such as illustrated in FIGS. 2 and 3. The formatting is considered optional as the extracted IR may already be in a format suitable for post processing by an LLM or other mechanisms, as discussed above. The process proceeds to block 430 of FIGS. 4 and 5 to continue with the patient summary generation process.

The techniques disclosed herein enables the summarizing of a range of information the provider needs for a single medication, class, or category of medications for an individual patient, such as in an ambulatory setting. The technical approach of triggering a medication summary generation process, automatically or intentionally, selecting from a variety of data sources, extracting and processing relevant information from the selected sources, filtering and harmonizing the information to be fed into an LLM, then generating medication summary in view of a given patient’s specific conditions, on the fly in a readily updatable manner, is beyond the capabilities of any single or even a group of healthcare providers equipped with electronic health record systems. The presently disclosed techniques enable consideration of a huge amount of data residing in multiple databases with disparate data formats such as, and not limited to, past and home medications related to a given medication or class of medications, historical prescriptions in the same category (e.g., different statins), including dosage changes; medications tried and failed in lieu of the medication being newly considered; medication change due to adverse reactions, insurance reasons or prohibitive cost reasons; and many others.

In embodiments, the medication summary disclosed herein is focused on summarizing all the information the provider needs for a single medication, class, or category of medications for an individual patient, in an readily updatable manner. By streamlining this process, physicians are relieved of the burden of manually compiling data, empowering them to allocate their time more effectively in patient care. The medication summary generation techniques disclosed herein may be useful in several points in various workflows, such as in responding to online messaging queries from patients regarding specific medications or prescriptions

The developed approach described herein addresses these challenges and others by providing techniques for assisting healthcare providers with necessary and time-consuming and often tedious tasks. Techniques are disclosed herein for improving the efficiency of and reducing the computing resources required to perform various healthcare services in a clinical environment. In certain embodiments, techniques are disclosed for equipping a healthcare provider end user with a clinical software application that can be installed on and utilized from one or both of a mobile computing device and a desktop computing device to facilitate performance of the various tasks typically rendered by a healthcare provider as part of providing healthcare services to patients.

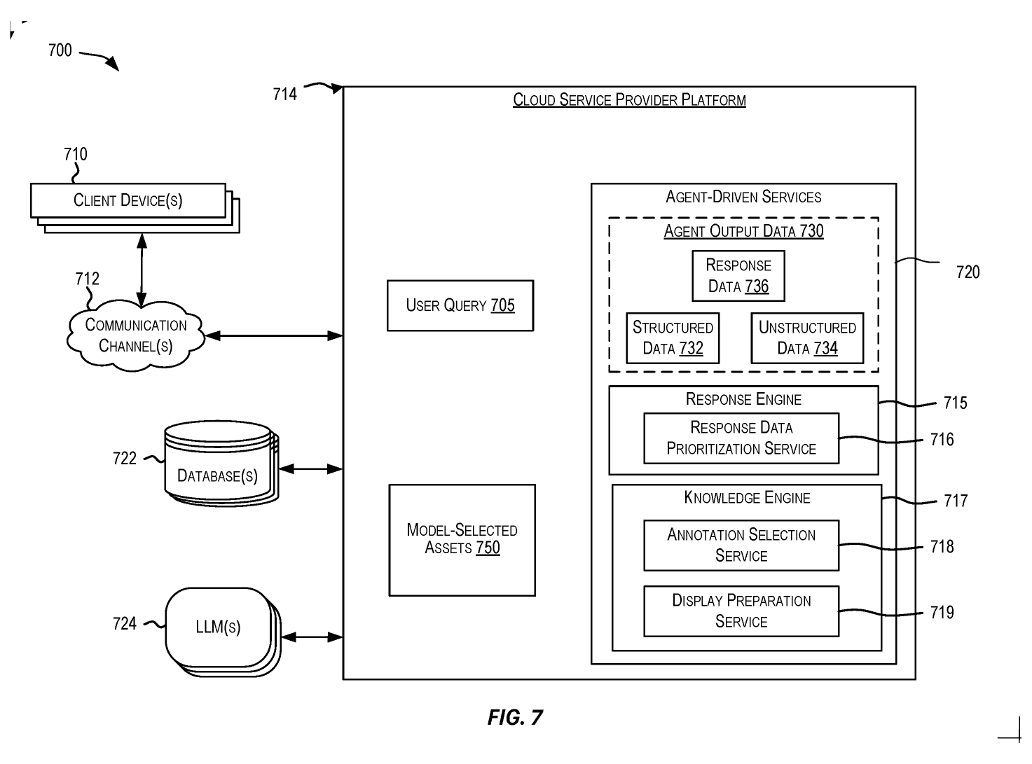

FIG. 7 shows a simplified diagram depicting a computing environment 700 incorporating an agent-driven digital assistant system configured to generate knowledge-grounded response data according to certain embodiments. As shown in FIG. 7, the computing environment 700 includes one or more client devices 710 (hereinafter “client devices 710”), one or more communication channels 712 (hereinafter “communication channels 712”), a cloud service provider platform 714 (hereinafter “platform 714”), one or more databases 722 (hereinafter “databases 722”), and one or more LLMs 724 (hereinafter “LLMs 724”). The platform 714, which can be included as part of a cloud infrastructure of a cloud service provider (e.g., Oracle Cloud Infrastructure or OCI), can be configured to communicate with, send data and information to, and receive data and information from the client devices 710 via the communication channels 712. Additionally, the platform 714 can be configured to access and/or call the databases 722 and the LLMs 724 to obtain and/or receive data and information from the databases 722 and the LLMs 724. Data and information received from the client devices 710, the databases 722, and the LLMs 724 can be used by the platform 714 to execute tasks and perform services such as automatically generating one or more portions of knowledge-grounded response data. While FIG. 7 shows the databases 722 and the LLMs 724 as being separate from the platform 714, this is not intended to be limiting, and one or more of the databases 722 and/or one or more of the LLMs 724 can be included as part of the platform 714 and/or the cloud infrastructure in which the platform 714 is included. While FIG. 7 describes the computing environment 700 as including the LLMs 724, other types of ML models can be included in the computing environment 700, such as an ML model configured for analyzing audio data and/or generating text based on audio data or an ML model configured to generate an execution plan for a group of multiple agent-driven services (or sub-services) included in the platform 714.

Each client device included in the client devices 710 can be any kind of electronic device that is capable of: executing applications; presenting information textually, graphically, and audibly such as via a display and a speaker; collecting information via one or more sensing elements such as image sensors, microphones, tactile sensors, touchscreen displays, and the like; connecting to a communication channel such as the communication channels 712 or a network such as a wireless network, wired network, a public network, a private network, and the like, to send and receive data and information; and/or storing data and information locally in one or more storage mediums of the electronic device and/or in one or more locations that are remote from the electronic device such as a cloud-based storage system, the platform 714, and/or the databases 722. Examples of electronic devices include, and are not limited to, mobile phones, desktop computers, portable computing devices, computers, workstations, laptop computers, tablet computers, and the like.

In some implementations, an application can be installed on, executing on, and/or accessed by a client device included in the client devices 710. The application and/or a user interface of the application can be utilized and/or interacted with (e.g., by an end user) to access, utilize, and/or interact with one or more services provided by the platform 714. The client devices 710 can be configured to receive multiple forms of input such as touch, text, voice, and the like, and the application can be configured to transform that input into one or more messages which can be transmitted or streamed to the platform 714 using one or more communication channels of the communication channels 712. Additionally, the client device can be configured to receive messages, data, and information from the platform 714 using one or more communication channels of the communication channels 712 and the application can be configured to present and/or render the received messages, data, and information in one or more user interfaces of the application. In some cases, the platform 714 receives one or more user queries, such as a user query 705, from the client devices 710. In some cases, the platform 714 provides one or more knowledge-grounded responses, such as knowledge-grounded response data 790, to the client devices 710.

Each communication channel included in the communication channels 712 can be any kind of communication channel that is capable of facilitating communication and the transfer of data and/or information between one or more entities such as the client devices 710, the platform 714, the databases 722, and the LLMs 724 (or other ML models). Examples of communication channels include, and are not limited to, public networks, private networks, the Internet, wireless networks, wired networks, fiber optic networks, local area networks, wide area networks, and the like. The communication channels 712 can be configured to facilitate data and/or information streaming between and among the one or more entities. In some implementations, data and/or information can be streamed using one or more messages and according to one or more protocols. Each of the one or more messages can be a variable length message and each communication channel included in the communication channels 712 can include a stream orchestration layer that can receive the variable length message in accordance with a predefined interface, such as an interface defined using an interface description language like AsyncAPI. Each of the variable length messages can include context information that can be used to determine the route or routes for the variable length message as well as a text or binary payload of arbitrary length. Each of the routes can be configured using a polyglot stream orchestration language that is agnostic to the details of the underlying implementation of the routing tasks and destinations.

Each database included in the databases 722 can be any kind of database that is capable of storing data and/or information and managing data and/or information. Data and/or information stored by each database can include data and/or information generated by, provided by, and/or otherwise obtained by the platform 714. Additionally, or alternatively, data and/or information stored and/or managed by each database can include data and/or information generated by, provided by, and/or otherwise obtained by other sources such as the client devices 710 and/or LLMs 724 (or other ML models). One or more databases that are included in the databases 722 can be part of a platform for storing and managing healthcare information such as electronic health records for patients, electronic records of healthcare providers, and the like, and can store and manage electronic health records for patients of healthcare providers. An example platform is the Oracle Health Millenium Platform. Additionally, one or more databases included in the databases 722 can be provided by, managed by, and/or otherwise included as part of a cloud infrastructure of a cloud service provider (e.g., Oracle Cloud Infrastructure or OCI). Data and/or information stored and/or managed by the databases 722 can be accessed using one or more application programming interfaces (APIs) of the databases 722.

Each LLM included in the LLMs 724 can be any kind of LLM that is capable of obtaining or generating or retrieving one or more results in response to one or more inputs such as one or more machine-learning prompts (hereinafter, “ML prompts” or “prompts”). ML prompts for obtaining or generating or retrieving results from the LLMs 724 can obtained from or generated by or retrieved from or accessed from the client devices 710, the databases 722, the platform 714, and/or one or more other sources such as the Internet. Each ML prompt can be configured to cause the LLMs 724 to perform one or more tasks, which causes one or more results to be provided or generated and the like. ML prompts for the LLMs 724 can be pre-generated (e.g., before they are needed for a particular task) and/or generated in real-time (e.g., without a delay noticeable to a human user). In some implementations, prompts for the LLMs 724 can be engineered to achieve a desired result or results manually and/or by one or more ML models. In some implementations, ML prompts for the LLMs 724 can be engineered on demand (i.e., in real-time and/or as needed) and/or at particular intervals (e.g., once per day, upon log in by authenticated user into the platform 714). Each ML prompt of the one or more ML prompts can include a request, such as a query, for a task to be performed by the LLMs 724. In some cases, an ML prompt can include additional information, such as data generated by one or more services (e.g., agent-driven services) included in the platform 714. The additional information can include information such as one or more ML prompt templates, structured data that is configured to be interpreted (e.g., semantically interpreted) by a computing system component (e.g., an ML model, an agent-driven service, etc.), unstructured data that is configured to be interpreted (e.g., semantically interpreted) by a human, responses from one or more ML models, output data generated by one or more agent-driven services, and/or other information suitable to include in an ML prompt. LLMs included in the LLMs 724 can be pre-trained, fine-tuned, open source, off-the-shelf, licensed, subscribed to, and the like. Additionally, LLMs included in the LLMs 724 can include or have any size context window (e.g., can accept any number of tokens) and can be capable of interpreting complex instructions. One or more LLMs included in the LLMs 724 can be provided by, managed by, and/or otherwise included as part of the platform 714 and/or a cloud infrastructure of a cloud service provider (e.g., Oracle Cloud Infrastructure or OCI) that supports the platform 714. One or more LLMs included in the LLMs 724 can be accessed using one or more APIs of the LLMs 724 and/or a platform hosting or supporting or providing the LLMs 724. In some implementations, one or more additional ML models included in the environment 700 may have one or more characteristics that are similar to characteristics described in regard to the LLMs 724.

The platform 714 can be configured to include various capabilities and provide various services to subscribers (e.g., end users) of the various services. In some implementations, such as in the case of an end user or subscriber being a healthcare provider, the healthcare provider can utilize the various services to facilitate the observation, care, treatment, management, and so on of their patient populations. For example, a healthcare provider can utilize the functionality provided by the various services provided by the platform 714 to examine and/or treat and/or facilitate the examination and/or treatment of a patient; view, edit, and/or manage a patient’s electronic health record; perform administrative tasks such as placing medical orders, scheduling appointments, managing patient populations, providing customer service to facilitate operation of a healthcare environment in which the healthcare provider practices, and so on.

In some implementations, the services provided by the platform 714 can include, and are not limited to, a response engine 715 and a knowledge engine 717. In some implementations, one or more services provided by the platform 714, such as the response engine 715 and/or the knowledge engine 717, can be configured to operate as agent-driven services, such as agent-driven services 720. In some implementations, an execution plan guides activities of one or more of the agent-driven services 720 provided by the platform 714. For example, the platform 714 can include, such as included in or in addition to the LLMs 724, a generative AI model (or another suitable ML model included in the environment 700) that is configured to generate (or modify) an execution plan. In this example, the execution plan can describe actions associated with one or more of the agent-driven services 720 provided by the platform 714. Based on the execution plan generated by the example generative AI model, one or more of the agent-driven services 720 can be configured to operate and/or interact with one or more additional ones of the agent-driven services 720. In some implementations, an output of the platform 714 is described by the execution plan. For example, the example generative AI model could be configured to generate an execution plan based on request data associated with the platform 714 (such as request data included in at least one query received by the platform 714 from one or more of the client devices 710). In some cases, the platform 714 and/or the example generative AI model can determine that the request data is associated with at least one agent-driven service of the services 720 provided by the platform 714, such as request data associated with the response engine 715 and/or the knowledge engine 717. In this example, the example generative AI model can generate an execution plan that describes one or more agent tasks for the at least one agent-driven service provided by the platform 714, such as agent tasks for generating a response to a user query and/or generating knowledge-grounded response data. For example, the response engine 715, the knowledge engine 717, and/or additional services in the agent-driven services 720 generates at least one response data object, such as the knowledge-grounded response data 790, based on a combination of multiple data outputs from the response engine 715 and/or the knowledge engine 717. In this example, the knowledge engine 717 generates the knowledge-grounded response data 790 by combining multiple data outputs from the response engine 715 and/or the knowledge engine 717 in a combination that is described by the execution plan.

In some implementations, the generated execution plan can omit instructions for implementing an agent task and include data outlining an agent task (e.g., data outlining one or more data sources, inputs, or requested outputs). In some implementations, one or more service of the agent-driven services 720 can generate its own instructions for implementing an agent task based on the data outlined in the execution plan. For example, an agent-driven service included in (or otherwise associated with) the response engine 715 can construct one or more ML prompts for generating response data, such as by using data outlined in the example execution plan to identify a prompt template (e.g., from a library of templates), a data source (e.g., a data repository storing information related to the user query 705), and one or more of the LLMs 724 (e.g., configured to generate text data summarizing medical information related to the user query 705). As another example, an agent-driven service included in (or otherwise associated with) the knowledge engine 717 can construct one or more ML prompts for generating and/or annotating the knowledge-grounded response data 790, such as by using data outlined in the example execution plan to identify a prompt template, a data source (e.g., data summarized by the response engine 715 and/or the data repository storing information related to the user query 705), and one or more of the LLMs 724 (e.g., configured to determine at least one response annotation using the data summarized by the response engine 715). In some cases, based on the data outlined in the execution plan, the response engine 715 and/or the knowledge engine 717 can be configured to generate respective instructions by which the services 716 and/or 718 can operate and/or interact with one or more additional services provided by the platform 714 (such as, and not limited to, additional services of the agent-driven services 720). Examples of skill-driven and LLM-based and agent-driven digital assistants are described in U.S. Patent Application No. 17,648,376, filed on January 19, 2022, and U.S. Patent Application No. 18/624,472, filed on April 2, 2024, each of which are incorporated by reference as if fully set forth herein.

In the platform 714, one or more of the agent-driven services 720 are configured, such as based on one or more generated execution plans, to create and/or annotate one or more portions of the knowledge-grounded response data 790. In the computing environment 700, the agent-driven services 720 can create the knowledge-grounded response data 790 in response to receiving one or more queries, such as a user query 705. To create the knowledge-grounded response data 790, the agent-driven services 720 perform, via the platform 714, one or more of acquiring LLMs, execution plan creation and/or implementation, asset identification (such as identification of one or more model-selected assets 750), and providing the knowledge-grounded response data 790 to one or more additional computing systems, such as to the client devices 710. For example, the platform 714 may receive the user query 705 from a particular one of the client devices 710. In addition, the platform 714 may generate at least one execution plan based on the user query 705. In some cases, one or more of the response engine 715, the knowledge engine 717, or one or more additional services of the agent-driven services 720 may identify at least one of the LLMs 724 based on the execution plan (or respective portions of the execution plan). In addition, one or more of the response engine 715, the knowledge engine 717, or the one or more additional services of the agent-driven services 720 may identify, such as from the databases 722, at least one asset based on the execution plan (or respective portions of the execution plan). Based on the identified one(s) of the LLMs 724 and/or the identified asset(s), one or more of the response engine 715, the knowledge engine 717, or the one or more additional services of the agent-driven services 720 may generate and/or modify the knowledge-grounded response data 790. In some cases, the knowledge-grounded response data 790 includes a combination of response data and attention cue data, such as response data that responds to a question (or other query type) included in the user query 705 and attention cue data that draws a user’s attention to at least a portion of the response data. Examples of response data can include text data, numeric data, image data (e.g., a radiology image), tabulated data (e.g., arranged in a table or other suitable format), or other types of response data suitable for responding to a user query. Examples of attention cue data can include highlighting data (e.g., color text, color background, color-vision deficiency patterns, etc.), font data (e.g., font size, italics, bold, underlining, typeface, etc.), audio data (e.g., automatic speech generation, audible alert data, etc.), haptic data (e.g., vibration, etc.), or other suitable types of attention cue data suitable for drawing user attention to at least a portion of response data.

In some implementations, the response engine 715 can be configured to automatically generate some or all response data that is included in the knowledge-grounded response data 790. For example, by utilizing an execution plan that is generated based on the user query 705, the response engine 715 may identify a first LLM from the LLMs 724 and one or more assets from the databases 722, such as one or more of an asset 750A, an asset 750B, through an asset 750N that are included in the model-selected assets 750. In addition, the response engine 715 may generate one or more ML prompts based on the one or more assets and provide the one or more ML prompts to the first LLM. Based on information received from the first LLM (e.g., in response to the one or more ML prompts), the response engine 715 may generate response data that responds to a question included in the user query 705. For example, if the user query 705 includes a question “How has Ms. Henderson’s new blood pressure medication been working?” the response engine 715 may identify, such as from an electronic health record (hereinafter, “EHR”) associated with the patient Ms. Henderson, a group of blood pressure measurements from a time period associated with a blood pressure medication currently prescribed to the patient. The response engine 715 may select the group of blood pressure measurements as information included in the asset 750A. In some cases, the response engine 715 may identify one or more additional assets from the databases 722 and include the additional assets in the model-selected assets 750, such as including in the asset 750B information describing the currently prescribed blood pressure medication or including in the asset 750N information describing additional medical factors for the patient (e.g., an additional diagnosis, a preferred exercise frequency for the patient, etc.) Continuing with this example, the response engine 715 may determine that the first LLM is fine-tuned to summarize information. In addition, the response engine 715 may generate a first ML prompt that includes one or more of the identified assets (e.g., assets 750A through 750N) and provide the first ML prompt to the first LLM. Based on data received from the first LLM, e.g., data summarizing the identified assets included in the first ML prompt, the response engine 715 may generate response data that includes a combination of text and tabulated numeric data, such as a table of blood pressure measurements and a text description of a trend in the blood pressure measurements since the patient Ms. Henderson began taking the currently prescribed blood pressure medication.

In some implementations, the knowledge engine 717 can be configured to automatically generate some or all attention cue data that is included in the knowledge-grounded response data 790. For example, by utilizing the execution plan that is generated based on the user query 705, the knowledge engine 717 may identify a second LLM from the LLMs 724. In addition, the knowledge engine 717 may identify one or more assets, such as one or more the response data generated by the response engine 715 and/or one or more of the assets 750A through 750N. In some cases, the knowledge engine 717 may generate one or more ML prompts based on the one or more assets and provide the one or more ML prompts to the second LLM. Based on information received from the second LLM (e.g., in response to the one or more ML prompts), the knowledge engine 717 may generate attention cue data that draws user attention to at least a portion of the response data generated by the response engine 715. Continuing with the example question “How has Ms. Henderson’s new blood pressure medication been working?” the knowledge engine 717 may determine that the second LLM is fine-tuned to identify high-relevance data in one or more assets. In addition, the knowledge engine 717 may generate a second ML prompt that includes one or more of the identified assets (e.g., the response data generated by the response engine 715 and the assets 750A through 750N) and provide the second ML prompt to the second LLM. Based on data received from the second LLM, e.g., data identifying high-relevance data included in the second ML prompt, the knowledge engine 717 may generate attention cue data that draws attention to the high-relevance data, such as color highlighting that draws attention to a trend in the blood pressure measurements and a bold font style that draws attention to information describing a possible interaction of the currently prescribed blood pressure medication with an additional medication frequently prescribed for an additional diagnosis of the patient. In addition, the knowledge engine 717 may generate or modify the knowledge-grounded response data 790 to include the attention cue data, e.g., modifying the knowledge-grounded response data 790 to apply the color highlighting to at least a portion of the tabulated numeric data and the bold font style to at least a portion of the text data. In some cases, the knowledge engine 717 may modify the knowledge-grounded response data 790 to include additional response data, such as interactive reference data (e.g., a URL address) that provides one or more references describing a source of information included in the knowledge-grounded response data 790. Continuing with the above example, the knowledge engine 717 may generate first interactive reference data that provides a first reference to one or more EHRs including blood pressure measurements for the patient. In addition, the knowledge engine 717 may generate second interactive reference data that provides a second reference to medication information (e.g., a medication reference database) describing possible interactions of the currently prescribed blood pressure medication.

In some implementations, the platform 714 (or a component thereof) may provide the knowledge-grounded response data 790 to one or more additional computing systems. For example, the platform 714 may identify a particular client device of the client devices 710 from which the user query 705 was received. In addition, the platform 714 may provide the knowledge-grounded response data 790 to the particular client device. In some cases, the particular client device is configured to perform one or more operations based on the knowledge-grounded response data 790, such as operations related to displaying the combination of the response data and the attention cue data included in the knowledge-grounded response data 790. For example, the particular client device may be configured to display the response data as annotated by the attention cue data, e.g., the table of blood pressure measurements and the text description as annotated by the color highlighting, the bold font style, and the interactive reference data. In addition, the particular client device may be configured to receive additional input data based on the knowledge-grounded response data 790, such as a user input indicating a selection of at least a portion of the knowledge-grounded response data 790. For example, responsive to receiving a user selection input of the first interactive reference data, the particular client device may be configured to send, to the platform 714, a request to receive at least a portion of the first reference, such as the one or more EHRs (or a portion thereof) including blood pressure measurements for the patient. In addition, responsive to receiving an additional user selection input of the second interactive reference data, the particular client device may be configured to send, to the platform 714, an additional request to receive at least a portion of the second reference, such as the medication information (or a portion thereof) describing possible interactions of the currently prescribed blood pressure medication. In some cases, the data architecture and/or configuration of the platform 714, such as the combination of the response engine 715 and the knowledge engine 717 and/or combination of some or all described features thereof, can improve user comprehension of information provided in response to user queries, such as the user query 705. For example, the combination of the response data with the attention cue data, such as included in the knowledge-grounded response data 790, can improve comprehension or reduce reading time by a user, such as by drawing the user’s attention to portions of the response data that are annotated by the attention cue data. In addition, the combination of the response data with the interactive reference data that is included in the attention cue data, such as included in the knowledge-grounded response data 790, can improve user trust in the response data by facilitating fast identification of potentially inaccurate data (e.g., hallucinations) generated by one or more of the LLMs 724, such as by providing fast access to reference information via the interactive reference data.

In FIG. 7, the response engine 715 and the knowledge engine 717 are described as utilizing a particular execution plan (e.g., respective portions of a same execution plan), and other implementations are possible. For example, a cloud service provider platform may generate a respective execution plan for each particular agent-driven service that is included in (or otherwise utilized by) the example cloud service provider platform. In FIG. 7, the response engine 715 and the knowledge engine 717 are described as respectively identifying the first LLM and the second LLM from the LLMs 724, and other implementations are possible. For example, in various instances, the response engine 715 and the knowledge engine 717 (or others of the agent-driven services 720) may identify from the LLMs 724 at least one same LLM, at least one different LLM, and/or a combination of different and same LLMs.

In some instances, the agent-driven services 720 can be utilized to access pre-trained and/or fine-tuned ML models, such as one or more of the LLMs 724. The pre-trained ML models serve as foundational elements, possessing extensive language understanding derived from vast datasets. This capability enables the models to generate coherent responses across various topics, facilitating transfer learning. Pre-trained models offer cost-effectiveness and flexibility, which allows for scalable improvements and continuous pre-training with new data, often establishing benchmarks in natural language processing tasks. Conversely, fine-tuned models are specifically trained for tasks or industries (e.g., plan creation utilizing the LLM's in-context learning capability, knowledge or information retrieval on behalf of an agent, response generation for human-like conversation, etc.), enhancing their performance on specific applications and enabling efficient learning from smaller, specialized datasets. Fine-tuning provides advantages such as task specialization, data efficiency, quicker training times, model customization, and resource efficiency. In some embodiments, fine-tuning may be particularly advantageous for niche applications and ongoing enhancement. In other instances, the agent-driven services 720 can be utilized to pre-train and/or fine-tune the LLMs. The agent-driven services 720, or any subset thereof, may be standalone or part of a machine-learning operationalization framework, inclusive of hardware components like processors (e.g., CPU, GPU, TPU, FPGA, or any combination), memory, and storage. This framework operates software or computer program instructions (e.g., TensorFlow, PyTorch, Keras, etc.) to execute arithmetic, logic, input/output commands for training, validating, and deploying machine-learning models in a production environment. In certain instances, the agent-driven services 720 implement the training, validating, and deploying of the models using a cloud platform such as Oracle Cloud Infrastructure (OCI). In some cases, leveraging a cloud platform can make machine-learning more accessible, flexible, and cost-effective, which can facilitate faster model development and deployment for developers.

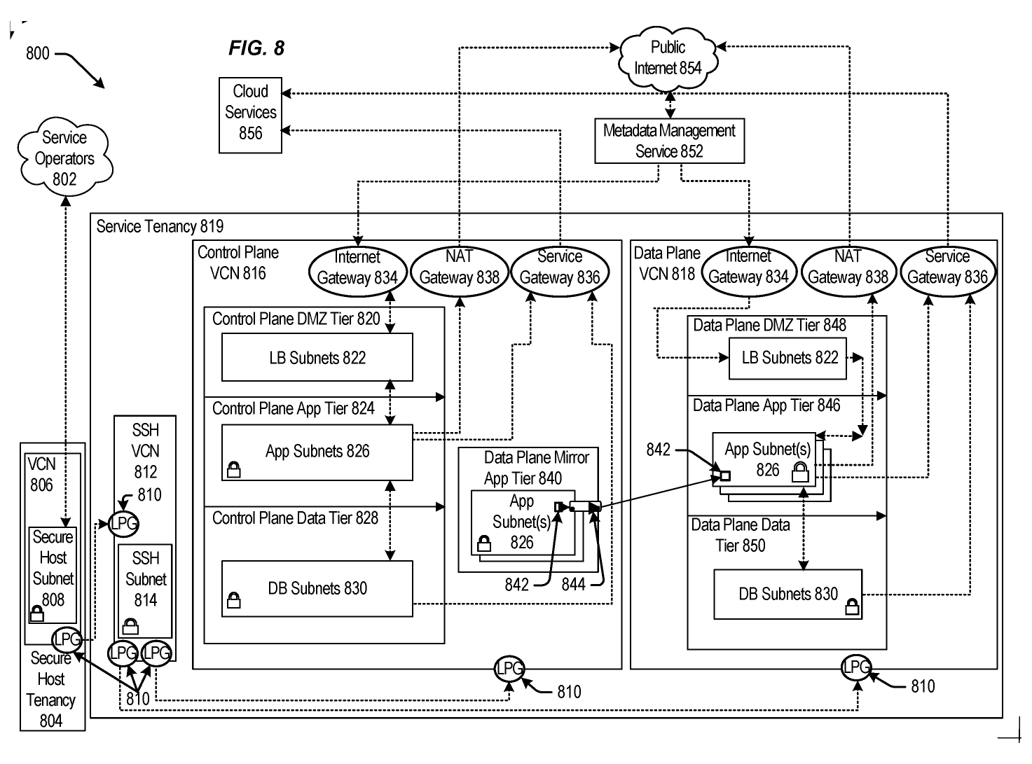

Although not shown, the platform 714 can include other capabilities and services such as authentication services, management services, task management services, notification services, and the like. The various capabilities and services of the platform 714 can be implemented utilizing one or more computing resources and/or servers of the platform 714 and provided by the platform 714 by way of subscriptions. Additionally, or alternatively, while FIG. 7 shows the services of the platform 714 as being separate services, one or more of the services can be combined with other services and/or be considered to be a sub-service of another service. In some implementations, a particular service in the agent-driven services 720 may utilize an output from another service in the agent-driven services 720, such as to facilitate quick completion of one or more operations by the particular service. For example, as shown in FIG. 8, one or more of the response engine 715 or the knowledge engine 717 can include at least one sub-service. In addition, one or more of the response engine 715 or the knowledge engine 717 can access one or more outputs from one or more services of the agent-driven services 720, such as agent output data 730. In some cases, the availability of the agent output data 730 to multiple services in the agent-driven services 720, such as at least the response engine 715 and the knowledge engine 717, can improve response time by the multiple services in the agent-driven services 720. For example, the response engine 715 and the knowledge engine 717 may provide one or more outputs with decreased response time and decreased use of computing resources (e.g., processing resources, memory resources, ML model resources, etc.) by using some or all of the agent output data 730 as input data.

FIG. 7 depicts the structured data 732 and the unstructured data 734 as being included in the agent output data 730, and other implementations are possible, such as one or more of the data 732 or 734 being included in the model-selected assets 750. In some cases, one or more of the structured data 732 and the unstructured data 734 is an output from one or more additional services of the agent-driven services 720, such as additional services configured for generating text data based on audio data (e.g., transcription of spoken conversation between a patient and a healthcare provider), identifying EHRs for a particular patient, or other tasks suitable to be performed by the agent-driven services 720. In FIG. 7, the structured data 732 can include data that is structured to be interpreted by a computing device, such as database records, JavaScript data objects, or other data objects (e.g., included in one or more EHRs) that are intended for computer interpretation (e.g., not intended for human interpretation). In FIG. 7, the unstructured data 734 can include data that lacks a structure interpreted by a computing device, such as patient appointment notes (e.g., Subjective, Objective, Assessment, and Plan notes, “SOAP notes”), medical reference materials (e.g., medication documentation, surgical procedure guidelines, etc.), or other types of information that are intended for human interpretation.

In the computing environment 700, the response engine 715 can include at least one sub-service, such as a response data prioritization service 716. In some cases, the response data prioritization service 716 may access at least one of the agent output data 730, such as one or more of the structured data 732 or the unstructured data 734. In addition, the response data prioritization service 716 may evaluate portions of the structured data 732 or the unstructured data 734 for potential inclusion in response data. For example, the response data prioritization service 716 may evaluate a data portion to determine whether the data is relevant to a question or other information in the user query 705. In some cases, the response data prioritization service 716 may send one or more portions of the structured data 732 or the unstructured data 734 to at least one LLM of the LLMs 724, such as in an ML prompt. In some implementations, the ML prompt also includes data corresponding to the user query 705, such as a question included in the user query 705 or additional data (e.g., determined by a service in the agent-driven services 720), such as additional data corresponding to the user query 705.

In the computing environment 700, the response data prioritization service 716 may receive, from the at least one LLM, multiple data portions that are extracted from one or more of the structured data 732 or the unstructured data 734. In addition, the response data prioritization service 716 may determine a relevance status for each of the multiple data portions, such as by comparing each data portion to one or more relevance threshold values. In some cases, the response data prioritization service 716 may determine, such as by determining a similarity (e.g., semantic similarity) between each data portion and query data (e.g., information associated with the user query 705), whether each data portion exceeds (or fulfill another relationship with) the one or more relevance threshold values. For example, based on a comparison to a first relevance threshold value, the response data prioritization service 716 may identify various data portions as high-relevance data, such as high-relevance data that directly answers one or more questions included in the user query 705. In some cases, the response data prioritization service 716 may select one or more of the LLMs 724 to modify some of the high-relevance data before inclusion in response data 736. For example, the response data prioritization service 716 may provide a first portion of the high-relevance data to a first LLM (e.g., from the LLMs 724) that is fine-tuned to summarize received information in text summary data. Examples of text summary data can include paragraphs, single sentences, or other human-readable text data that summarizes larger amounts of information. In addition, the response data prioritization service 716 may modify the response data 736 to include the text summary data which summarizes the high-relevance data. As another example, the response data prioritization service 716 may provide a second portion of the high-relevance data to a second LLM (e.g., from the LLMs 724) that is fine-tuned to arrange received information as tabulated data, such as multiple items of information that are intended to be interpreted as a group. Examples of tabulated data can include tables, bulleted lists, numbered lists, or other organized arrangements of multiple items of information intended to be interpreted as a group. Examples of information that could be arranged as tabulated data can include a group of lab results, a group of comparison medications (e.g., generics, non-generics, etc.), a group of potential side effects of a surgical procedure, or other groups of information items. In addition, the response data prioritization service 716 may modify the response data 736 to include the tabulated data in which the high-relevance data is arranged.

In some implementations, based on a comparison to a second relevance threshold value, the response data prioritization service 716 may identify one or more data portions (e.g., extracted from one or more of the structured data 732 or the unstructured data 734) as medium-relevance data. In some cases, the medium-relevance data can include supplemental data that does not directly answer one or more questions included in the user query 705 and which provides additional data about a topic identified in the one or more questions. Examples of supplemental data can include information about a diagnosed condition, information about a patient circumstance (e.g., a high-exercise lifestyle, a preference to avoid injected medications, etc.), or other types of information that are generally related to a question. In some cases, the response data prioritization service 716 may select one or more of the LLMs 724 to modify some of the medium-relevance data before inclusion in the response data 736 . For example, the response data prioritization service 716 may provide a portion of the medium-relevance data to the first LLM that is fine-tuned to summarize received information in text summary data. In addition, the response data prioritization service 716 may modify the response data 936 to include additional text summary data which summarizes the medium-relevance data.

In the computing environment 700, the knowledge engine 717 can include at least one sub-service, such as one or more of an annotation selection service 718 or a display preparation service 719. In some cases, one or more of the annotation selection service 718 or the display preparation service 719 may access at least one of the agent output data 730, such as the response data 736 that is generated by the response engine 715. Examples of computer-implemented instructions related to display preparation service can include hypertext markup language (HTML) instructions, extensible markup language (XML) instructions, or other suitable types of instructions for implementing data display (e.g., visual display, audio display, etc.) via one or more user interface devices.

In the computing environment 700, the annotation selection service 718 may provide one or more portions of the response data 736 to at least one LLM of the LLMs 724, such as in an ML prompt. In some implementations, the ML prompt also includes additional data (e.g., determined by a service in the agent-driven services 720), such as additional data corresponding to one or more of the user query 705, the structured data 732, or the unstructured data 734. For example, the at least one LLM may be fine-tuned to identify, in the response data 736, one or more portions of data that have a relatively high similarity to data included in one or more of the user query 705, the structured data 732, or the unstructured data 734, such as a portion of high-relevance text summary data in the response data 736 that has a high similarity to text data of a question included in the user query 705. In some cases, the annotation selection service 718 may receive, from the at least one LLM, data identifying at least one portion of the response data 736 for annotation. In addition, the annotation selection service 718 may determine one or more types of annotations to apply to the identified portion of the response data 736, such as annotations for highlighting, font styles, or other types of annotations that can be applied to response data. In some implementations, the annotation selection service 718 may determine at least one type of annotation that applies interactive reference data to the identified portion of the response data 736. For example, the annotation selection service 718 may determine one or more sources for the identified portion of the response data 736, such as a source document and/or source database associated with one or more of the structured data 732 or the unstructured data 734. Based on the determined one or more sources, the annotation selection service 718 may generate interactive reference data that indicates the source(s) for the identified portion of the response data 736. In some cases, the annotation selection service 718 may identify source address data associated with the source(s) for the identified portion of the response data 736. Examples of source address data can include a network address (e.g., a URL, a MAC address), computing component identification data (e.g., identification of a particular database, etc.), document identification data (e.g., identification of a particular document, identification of a section within a document, etc.), or other types of address data that can identify a location (or other identification type) for a source repository.

In the computing environment 700, the display preparation service 719 may identify one or more associated portions of response data. In addition, the display preparation service 719 may generate one or more computer-implemented instructions that combine the associated portions of response data for presentation via one or more user interface devices. In addition, the display preparation service 719 may generate at least one computer-implemented instruction that combines the associated portions, such as an HTML instruction (or other suitable instruction type) that applies a bold typeface to the sentence. As another example, the display preparation service 719 may determine an additional association between a second portion of the response data 736 that indicates tabulated data, such as a set of blood pressure measurements, and an additional annotation including interactive reference data, such as a patient chart that is a source document for the set of blood pressure measurements. In addition, the display preparation service 719 may generate at least one additional computer-implemented instruction that combines the additional associated portions, such as an additional HTML instruction (or other suitable instruction type) that applies an interactive link to the tabulated set of blood pressure measurements, e.g., the interactive link is directed to the patient chart. The computing environment 700 depicted in FIG. 7 is merely exemplary and not intended to unduly limit the scope of claimed embodiments. One of ordinary skill in the art would recognize many possible variations, alternatives, and modifications. For example, in some implementations, the computing environment 700900 can be implemented using more or fewer services than those shown in FIGS. 7, may combine two or more services, or may have a different configuration or arrangement of services.

### Examples of Cloud Infrastructure Architectures

The term cloud service is generally used to refer to a service that is made available by a cloud service provider (CSP) to users (e.g., cloud service customers) on demand (e.g., via a subscription model) using systems and infrastructure (cloud infrastructure) provided by the CSP. Typically, the servers and systems that make up the CSP’s infrastructure are separate from the user’s own on-premise servers and systems. Users can thus avail themselves of cloud services provided by the CSP without having to purchase separate hardware and software resources for the services. Cloud services are designed to provide a subscribing user easy, scalable access to applications and computing resources without the user having to invest in procuring the infrastructure that is used for providing the services.

There are several cloud service providers that offer various types of cloud services. As discussed herein, there are various types or models of cloud services including IaaS, software as a service (SaaS), platform as a service (PaaS), and others. A user can subscribe to one or more cloud services provided by a CSP. The user can be any entity such as an individual, an organization, an enterprise, and the like. When a user subscribes to or registers for a service provided by a CSP, a tenancy or an account is created for that user. The user can then, via this account, access the subscribed-to one or more cloud resources associated with the account.

As noted above, infrastructure as a service (IaaS) is one particular type of cloud computing. IaaS can be configured to provide virtualized computing resources over a public network (e.g., the Internet). In an IaaS model, a cloud computing provider can host the infrastructure components (e.g., servers, storage devices, network nodes (e.g., hardware), deployment software, platform virtualization (e.g., a hypervisor layer), or the like). In some cases, an IaaS provider may also supply a variety of services to accompany those infrastructure components (example services include billing software, monitoring software, logging software, load balancing software, clustering software, etc.). Thus, as these services may be policy-driven, IaaS users may be able to implement policies to drive load balancing to maintain application availability and performance.

In some instances, IaaS customers may access resources and services through a wide area network (WAN), such as the Internet, and can use the cloud provider's services to install the remaining elements of an application stack. For example, the user can log in to the IaaS platform to create virtual machines (VMs), install operating systems (OSs) on each VM, deploy middleware such as databases, create storage buckets for workloads and backups, and even install enterprise software into that VM. Customers can then use the provider's services to perform various functions, including balancing network traffic, troubleshooting application issues, monitoring performance, managing disaster recovery, etc.

In most cases, a cloud computing model will require the participation of a cloud provider. The cloud provider may, but need not be, a third-party service that specializes in providing (e.g., offering, renting, selling) IaaS. An entity might also opt to deploy a private cloud, becoming its own provider of infrastructure services.

In some examples, IaaS deployment is the process of putting a new application, or a new version of an application, onto a prepared application server or the like. It may also include the process of preparing the server (e.g., installing libraries, daemons, etc.). This is often managed by the cloud provider, below the hypervisor layer (e.g., the servers, storage, network hardware, and virtualization). Thus, the customer may be responsible for handling (OS), middleware, and/or application deployment (e.g., on self-service virtual machines (e.g., that can be spun up on demand)) or the like.

In some examples, IaaS provisioning may refer to acquiring computers or virtual hosts for use, and even installing needed libraries or services on them. In most cases, deployment does not include provisioning, and the provisioning may need to be performed first.

In some cases, there are two different challenges for IaaS provisioning. First, there is the initial challenge of provisioning the initial set of infrastructure before anything is running. Second, there is the challenge of evolving the existing infrastructure (e.g., adding new services, changing services, removing services, etc.) once everything has been provisioned. In some cases, these two challenges may be addressed by enabling the configuration of the infrastructure to be defined declaratively. In other words, the infrastructure (e.g., what components are needed and how they interact) can be defined by one or more configuration files. Thus, the overall topology of the infrastructure (e.g., what resources depend on which, and how they each work together) can be described declaratively. In some instances, once the topology is defined, a workflow can be generated that creates and/or manages the different components described in the configuration files.

In some examples, an infrastructure may have many interconnected elements. For example, there may be one or more virtual private clouds (VPCs) (e.g., a potentially on-demand pool of configurable and/or shared computing resources), also known as a core network. In some examples, there may also be one or more inbound/outbound traffic group rules provisioned to define how the inbound and/or outbound traffic of the network will be set up and one or more virtual machines (VMs). Other infrastructure elements may also be provisioned, such as a load balancer, a database, or the like. As more and more infrastructure elements are desired and/or added, the infrastructure may incrementally evolve.

In some instances, continuous deployment techniques may be employed to enable deployment of infrastructure code across various virtual computing environments. Additionally, the described techniques can enable infrastructure management within these environments. In some examples, service teams can write code that is desired to be deployed to one or more, but often many, different production environments (e.g., across various different geographic locations, sometimes spanning the entire world). However, in some examples, the infrastructure on which the code will be deployed must first be set up. In some instances, the provisioning can be done manually, a provisioning tool may be utilized to provision the resources, and/or deployment tools may be utilized to deploy the code once the infrastructure is provisioned.

FIG. 8 is a block diagram 800 illustrating an example pattern of an IaaS architecture, according to at least one embodiment. Service operators 802 can be communicatively coupled to a secure host tenancy 804 that can include a virtual cloud network (VCN) 806 and a secure host subnet 808. In some examples, the service operators 802 may be using one or more client computing devices, which may be portable handheld devices (e.g., an iPhone®, cellular telephone, an iPad®, computing tablet, a personal digital assistant (PDA)) or wearable devices (e.g., a Google Glass® head mounted display), running software such as Microsoft Windows Mobile®, and/or a variety of mobile operating systems such as iOS, Windows Phone, Android, BlackBerry 8, Palm OS, and the like, and being Internet, e-mail, short message service (SMS), Blackberry®, or other communication protocol enabled. Alternatively, the client computing devices can be general purpose personal computers including, by way of example, personal computers and/or laptop computers running various versions of Microsoft Windows®, Apple Macintosh®, and/or Linux operating systems. The client computing devices can be workstation computers running any of a variety of commercially-available UNIX® or UNIX-like operating systems, including without limitation the variety of GNU/Linux operating systems, such as for example, Google Chrome OS. Alternatively, or in addition, client computing devices may be any other electronic device, such as a thin-client computer, an Internet-enabled gaming system (e.g., a Microsoft Xbox gaming console with or without a Kinect® gesture input device), and/or a personal messaging device, capable of communicating over a network that can access the VCN 806 and/or the Internet.

The VCN 806 can include a local peering gateway (LPG) 810 that can be communicatively coupled to a secure shell (SSH) VCN 812 via an LPG 810 contained in the SSH VCN 812. The SSH VCN 812 can include an SSH subnet 814, and the SSH VCN 812 can be communicatively coupled to a control plane VCN 816 via the LPG 810 contained in the control plane VCN 816. Also, the SSH VCN 812 can be communicatively coupled to a data plane VCN 818 via an LPG 810. The control plane VCN 816 and the data plane VCN 818 can be contained in a service tenancy 819 that can be owned and/or operated by the IaaS provider.

The control plane VCN 816 can include a control plane demilitarized zone (DMZ) tier 820 that acts as a perimeter network (e.g., portions of a corporate network between the corporate intranet and external networks). The DMZ-based servers may have restricted responsibilities and help keep breaches contained. Additionally, the DMZ tier 820 can include one or more load balancer (LB) subnet(s) 822, a control plane app tier 824 that can include app subnet(s) 826, a control plane data tier 828 that can include database (DB) subnet(s) 830 (e.g., frontend DB subnet(s) and/or backend DB subnet(s)). The LB subnet(s) 822 contained in the control plane DMZ tier 820 can be communicatively coupled to the app subnet(s) 826 contained in the control plane app tier 824 and an Internet gateway 834 that can be contained in the control plane VCN 816, and the app subnet(s) 826 can be communicatively coupled to the DB subnet(s) 830 contained in the control plane data tier 828 and a service gateway 836 and a network address translation (NAT) gateway 838. The control plane VCN 816 can include the service gateway 836 and the NAT gateway 838.

The control plane VCN 816 can include a data plane mirror app tier 840 that can include app subnet(s) 826. The app subnet(s) 826 contained in the data plane mirror app tier 840 can include a virtual network interface controller (VNIC) 842 that can execute a compute instance 844. The compute instance 844 can communicatively couple the app subnet(s) 826 of the data plane mirror app tier 840 to app subnet(s) 826 that can be contained in a data plane app tier 846.

The data plane VCN 818 can include the data plane app tier 846, a data plane DMZ tier 848, and a data plane data tier 850. The data plane DMZ tier 848 can include LB subnet(s) 822 that can be communicatively coupled to the app subnet(s) 826 of the data plane app tier 846 and the Internet gateway 834 of the data plane VCN 818. The app subnet(s) 826 can be communicatively coupled to the service gateway 836 of the data plane VCN 818 and the NAT gateway 838 of the data plane VCN 818. The data plane data tier 850 can also include the DB subnet(s) 830 that can be communicatively coupled to the app subnet(s) 826 of the data plane app tier 846.

The Internet gateway 834 of the control plane VCN 816 and of the data plane VCN 818 can be communicatively coupled to a metadata management service 852 that can be communicatively coupled to public Internet 854. Public Internet 854 can be communicatively coupled to the NAT gateway 838 of the control plane VCN 816 and of the data plane VCN 818. The service gateway 836 of the control plane VCN 816 and of the data plane VCN 818 can be communicatively coupled to cloud services 856.

In some examples, the service gateway 836 of the control plane VCN 816 or of the data plane VCN 818 can make application programming interface (API) calls to cloud services 856 without going through public Internet 854. The API calls to cloud services 856 from the service gateway 836 can be one-way: the service gateway 836 can make API calls to cloud services 856, and cloud services 856 can send requested data to the service gateway 836. But, cloud services 856 may not initiate API calls to the service gateway 836.

In some examples, the secure host tenancy 804 can be directly connected to the service tenancy 819, which may be otherwise isolated. The secure host subnet 808 can communicate with the SSH subnet 814 through an LPG 810 that may enable two-way communication over an otherwise isolated system. Connecting the secure host subnet 808 to the SSH subnet 814 may give the secure host subnet 808 access to other entities within the service tenancy 819.

The control plane VCN 816 may allow users of the service tenancy 819 to set up or otherwise provision desired resources. Desired resources provisioned in the control plane VCN 816 may be deployed or otherwise used in the data plane VCN 818. In some examples, the control plane VCN 816 can be isolated from the data plane VCN 818, and the data plane mirror app tier 840 of the control plane VCN 816 can communicate with the data plane app tier 846 of the data plane VCN 818 via VNICs 842 that can be contained in the data plane mirror app tier 840 and the data plane app tier 846.

In some examples, users of the system, or customers, can make requests, for example create, read, update, or delete (CRUD) operations, through public Internet 854 that can communicate the requests to the metadata management service 852. The metadata management service 852 can communicate the request to the control plane VCN 816 through the Internet gateway 834. The request can be received by the LB subnet(s) 822 contained in the control plane DMZ tier 820. The LB subnet(s) 822 may determine that the request is valid, and in response to this determination, the LB subnet(s) 822 can transmit the request to app subnet(s) 826 contained in the control plane app tier 824. If the request is validated and requires a call to public Internet 854, the call to public Internet 854 may be transmitted to the NAT gateway 838 that can make the call to public Internet 854. Metadata that may be desired to be stored by the request can be stored in the DB subnet(s) 830.

In some examples, the data plane mirror app tier 840 can facilitate direct communication between the control plane VCN 816 and the data plane VCN 818. For example, changes, updates, or other suitable modifications to configuration may be desired to be applied to the resources contained in the data plane VCN 818. Via a VNIC 842, the control plane VCN 816 can directly communicate with, and can thereby execute the changes, updates, or other suitable modifications to configuration to, resources contained in the data plane VCN 818.

In some embodiments, the control plane VCN 816 and the data plane VCN 818 can be contained in the service tenancy 819. In this case, the user, or the customer, of the system may not own or operate either the control plane VCN 816 or the data plane VCN 818. Instead, the IaaS provider may own or operate the control plane VCN 816 and the data plane VCN 818, both of which may be contained in the service tenancy 819. This embodiment can enable isolation of networks that may prevent users or customers from interacting with other users’, or other customers’, resources. Also, this embodiment may allow users or customers of the system to store databases privately without needing to rely on public Internet 854, which may not have a desired level of threat prevention, for storage.

In other embodiments, the LB subnet(s) 822 contained in the control plane VCN 816 can be configured to receive a signal from the service gateway 836. In this embodiment, the control plane VCN 816 and the data plane VCN 818 may be configured to be called by a customer of the IaaS provider without calling public Internet 854. Customers of the IaaS provider may desire this embodiment since database(s) that the customers use may be controlled by the IaaS provider and may be stored on the service tenancy 819, which may be isolated from public Internet 854.

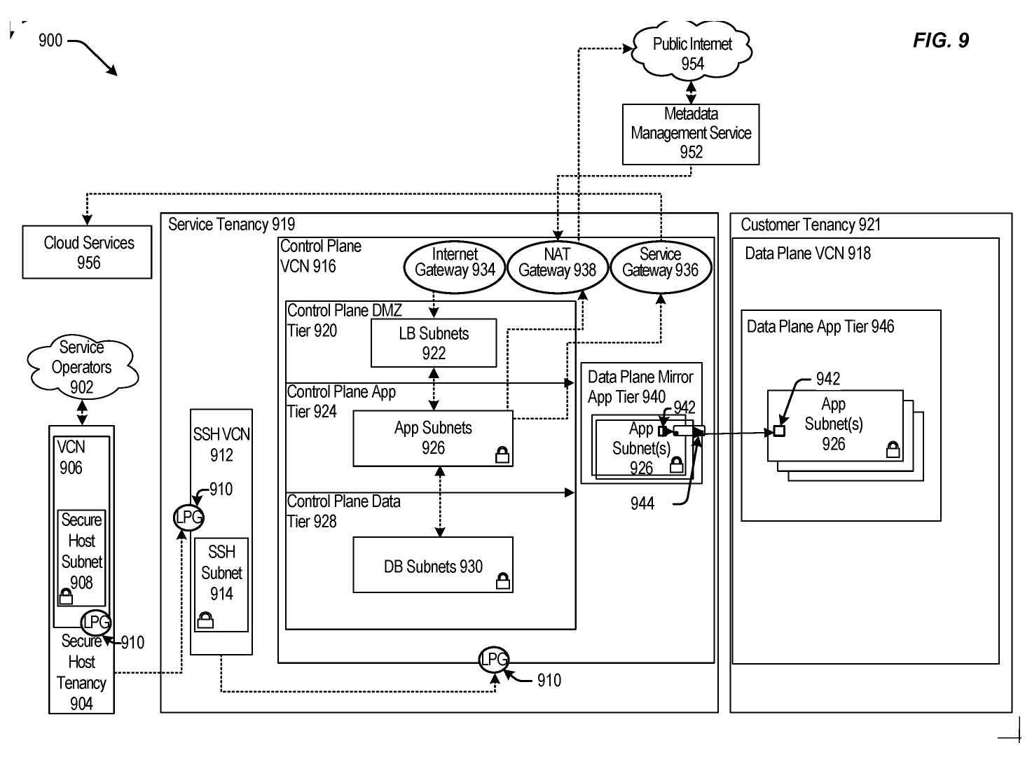

FIG. 9 is a block diagram 900 illustrating another example pattern of an IaaS architecture, according to at least one embodiment. Service operators 902 (e.g., service operators 802 of FIG. 8) can be communicatively coupled to a secure host tenancy 904 (e.g., the secure host tenancy 804 of FIG. 8) that can include a virtual cloud network (VCN) 906 (e.g., the VCN 806 of FIG. 8) and a secure host subnet 908 (e.g., the secure host subnet 808 of FIG. 8). The VCN 906 can include a local peering gateway (LPG) 910 (e.g., the LPG 810 of FIG. 8) that can be communicatively coupled to a secure shell (SSH) VCN 912 (e.g., the SSH VCN 812 of FIG. 8) via an LPG 810 contained in the SSH VCN 912. The SSH VCN 912 can include an SSH subnet 914 (e.g., the SSH subnet 814 of FIG. 8), and the SSH VCN 912 can be communicatively coupled to a control plane VCN 916 (e.g., the control plane VCN 816 of FIG. 8) via an LPG 910 contained in the control plane VCN 916. The control plane VCN 916 can be contained in a service tenancy 919 (e.g., the service tenancy 819 of FIG. 8), and the data plane VCN 918 (e.g., the data plane VCN 818 of FIG. 8) can be contained in a customer tenancy 921 that may be owned or operated by users, or customers, of the system.

The control plane VCN 916 can include a control plane DMZ tier 920 (e.g., the control plane DMZ tier 820 of FIG. 8) that can include LB subnet(s) 922 (e.g., LB subnet(s) 822 of FIG. 8), a control plane app tier 924 (e.g., the control plane app tier 824 of FIG. 8) that can include app subnet(s) 926 (e.g., app subnet(s) 826 of FIG. 8), a control plane data tier 928 (e.g., the control plane data tier 828 of FIG. 8) that can include database (DB) subnet(s) 930 (e.g., similar to DB subnet(s) 830 of FIG. 8). The LB subnet(s) 922 contained in the control plane DMZ tier 920 can be communicatively coupled to the app subnet(s) 926 contained in the control plane app tier 924 and an Internet gateway 934 (e.g., the Internet gateway 834 of FIG. 8) that can be contained in the control plane VCN 916, and the app subnet(s) 926 can be communicatively coupled to the DB subnet(s) 930 contained in the control plane data tier 928 and a service gateway 936 (e.g., the service gateway 836 of FIG. 8) and a network address translation (NAT) gateway 938 (e.g., the NAT gateway 838 of FIG. 8). The control plane VCN 916 can include the service gateway 936 and the NAT gateway 938.

The control plane VCN 916 can include a data plane mirror app tier 940 (e.g., the data plane mirror app tier 840 of FIG. 8) that can include app subnet(s) 926. The app subnet(s) 926 contained in the data plane mirror app tier 940 can include a virtual network interface controller (VNIC) 942 (e.g., the VNIC of 842) that can execute a compute instance 944 (e.g., similar to the compute instance 844 of FIG. 8). The compute instance 944 can facilitate communication between the app subnet(s) 926 of the data plane mirror app tier 940 and the app subnet(s) 926 that can be contained in a data plane app tier 946 (e.g., the data plane app tier 846 of FIG. 8) via the VNIC 942 contained in the data plane mirror app tier 940 and the VNIC 942 contained in the data plane app tier 946.

The Internet gateway 934 contained in the control plane VCN 916 can be communicatively coupled to a metadata management service 952 (e.g., the metadata management service 852 of FIG. 8) that can be communicatively coupled to public Internet 954 (e.g., public Internet 854 of FIG. 8). Public Internet 954 can be communicatively coupled to the NAT gateway 938 contained in the control plane VCN 916. The service gateway 936 contained in the control plane VCN 916 can be communicatively coupled to cloud services 956 (e.g., cloud services 856 of FIG. 8).

In some examples, the data plane VCN 918 can be contained in the customer tenancy 921. In this case, the IaaS provider may provide the control plane VCN 916 for each customer, and the IaaS provider may, for each customer, set up a unique compute instance 944 that is contained in the service tenancy 919. Each compute instance 944 may allow communication between the control plane VCN 916, contained in the service tenancy 919, and the data plane VCN 918 that is contained in the customer tenancy 921. The compute instance 944 may allow resources, that are provisioned in the control plane VCN 916 that is contained in the service tenancy 919, to be deployed or otherwise used in the data plane VCN 918 that is contained in the customer tenancy 921.

In other examples, the customer of the IaaS provider may have databases that live in the customer tenancy 921. In this example, the control plane VCN 916 can include the data plane mirror app tier 940 that can include app subnet(s) 926. The data plane mirror app tier 940 can reside in the data plane VCN 918, but the data plane mirror app tier 940 may not live in the data plane VCN 918. That is, the data plane mirror app tier 940 may have access to the customer tenancy 921, but the data plane mirror app tier 940 may not exist in the data plane VCN 918 or be owned or operated by the customer of the IaaS provider. The data plane mirror app tier 940 may be configured to make calls to the data plane VCN 918 but may not be configured to make calls to any entity contained in the control plane VCN 916. The customer may desire to deploy or otherwise use resources in the data plane VCN 918 that are provisioned in the control plane VCN 916, and the data plane mirror app tier 940 can facilitate the desired deployment, or other usage of resources, of the customer.

In some embodiments, the customer of the IaaS provider can apply filters to the data plane VCN 918. In this embodiment, the customer can determine what the data plane VCN 918 can access, and the customer may restrict access to public Internet 954 from the data plane VCN 918. The IaaS provider may not be able to apply filters or otherwise control access of the data plane VCN 918 to any outside networks or databases. Applying filters and controls by the customer onto the data plane VCN 918, contained in the customer tenancy 921, can help isolate the data plane VCN 918 from other customers and from public Internet 954.

In some embodiments, cloud services 956 can be called by the service gateway 936 to access services that may not exist on public Internet 954, on the control plane VCN 916, or on the data plane VCN 918. The connection between cloud services 956 and the control plane VCN 916 or the data plane VCN 918 may not be live or continuous. Cloud services 956 may exist on a different network owned or operated by the IaaS provider. Cloud services 956 may be configured to receive calls from the service gateway 936 and may be configured to not receive calls from public Internet 954. Some cloud services 956 may be isolated from other cloud services 956, and the control plane VCN 916 may be isolated from cloud services 956 that may not be in the same region as the control plane VCN 916. For example, the control plane VCN 916 may be located in “Region 1,” and cloud service “Deployment 8,” may be located in Region 1 and in “Region 2.” If a call to Deployment 8 is made by the service gateway 936 contained in the control plane VCN 916 located in Region 1, the call may be transmitted to Deployment 8 in Region 1. In this example, the control plane VCN 916, or Deployment 8 in Region 1, may not be communicatively coupled to, or otherwise in communication with, Deployment 8 in Region 2.

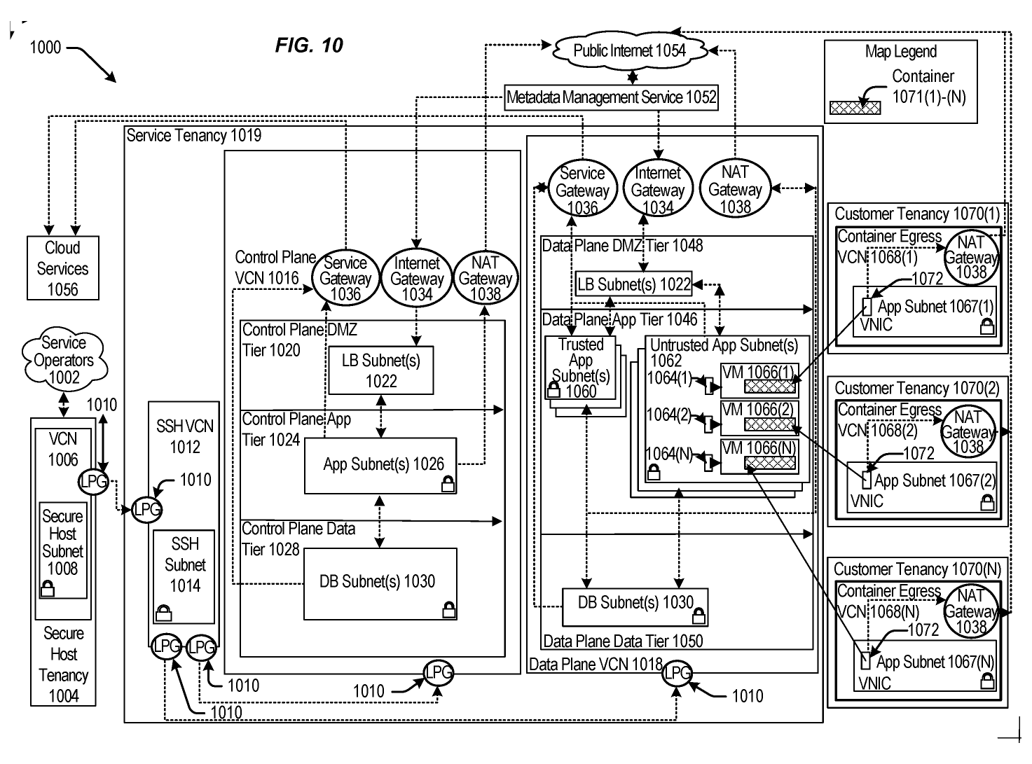

FIG. 10 is a block diagram 1000 illustrating another example pattern of an IaaS architecture, according to at least one embodiment. Service operators 1002 (e.g., service operators 802 of FIG. 8) can be communicatively coupled to a secure host tenancy 1004 (e.g., the secure host tenancy 804 of FIG. 8) that can include a virtual cloud network (VCN) 1006 (e.g., the VCN 806 of FIG. 8) and a secure host subnet 1008 (e.g., the secure host subnet 808 of FIG. 8). The VCN 1006 can include an LPG 1010 (e.g., the LPG 810 of FIG. 8) that can be communicatively coupled to an SSH VCN 1012 (e.g., the SSH VCN 812 of FIG. 8) via an LPG 1010 contained in the SSH VCN 1012. The SSH VCN 1012 can include an SSH subnet 1014 (e.g., the SSH subnet 814 of FIG. 8), and the SSH VCN 1012 can be communicatively coupled to a control plane VCN 1016 (e.g., the control plane VCN 816 of FIG. 8) via an LPG 1010 contained in the control plane VCN 1016 and to a data plane VCN 1018 (e.g., the data plane 818 of FIG. 8) via an LPG 1010 contained in the data plane VCN 1018. The control plane VCN 1016 and the data plane VCN 1018 can be contained in a service tenancy 1019 (e.g., the service tenancy 819 of FIG. 8).

The control plane VCN 1016 can include a control plane DMZ tier 1020 (e.g., the control plane DMZ tier 820 of FIG. 8) that can include load balancer (LB) subnet(s) 1022 (e.g., LB subnet(s) 822 of FIG. 8), a control plane app tier 1024 (e.g., the control plane app tier 824 of FIG. 8) that can include app subnet(s) 1026 (e.g., similar to app subnet(s) 826 of FIG. 8), a control plane data tier 1028 (e.g., the control plane data tier 828 of FIG. 8) that can include DB subnet(s) 1030. The LB subnet(s) 1022 contained in the control plane DMZ tier 1020 can be communicatively coupled to the app subnet(s) 1026 contained in the control plane app tier 1024 and to an Internet gateway 1034 (e.g., the Internet gateway 834 of FIG. 8) that can be contained in the control plane VCN 1016, and the app subnet(s) 1026 can be communicatively coupled to the DB subnet(s) 1030 contained in the control plane data tier 1028 and to a service gateway 1036 (e.g., the service gateway of FIG. 8) and a network address translation (NAT) gateway 1038 (e.g., the NAT gateway 838 of FIG. 8). The control plane VCN 1016 can include the service gateway 1036 and the NAT gateway 1038.

The data plane VCN 1018 can include a data plane app tier 1046 (e.g., the data plane app tier 846 of FIG. 8), a data plane DMZ tier 1048 (e.g., the data plane DMZ tier 848 of FIG. 8), and a data plane data tier 1050 (e.g., the data plane data tier 850 of FIG. 8). The data plane DMZ tier 1048 can include LB subnet(s) 1022 that can be communicatively coupled to trusted app subnet(s) 1060 and untrusted app subnet(s) 1062 of the data plane app tier 1046 and the Internet gateway 1034 contained in the data plane VCN 1018. The trusted app subnet(s) 1060 can be communicatively coupled to the service gateway 1036 contained in the data plane VCN 1018, the NAT gateway 1038 contained in the data plane VCN 1018, and DB subnet(s) 1030 contained in the data plane data tier 1050. The untrusted app subnet(s) 1062 can be communicatively coupled to the service gateway 1036 contained in the data plane VCN 1018 and DB subnet(s) 1030 contained in the data plane data tier 1050. The data plane data tier 1050 can include DB subnet(s) 1030 that can be communicatively coupled to the service gateway 1036 contained in the data plane VCN 1018.

The untrusted app subnet(s) 1062 can include one or more primary VNICs 1064(1)-(N) that can be communicatively coupled to tenant virtual machines (VMs) 1066(1)-(N). Each tenant VM 1066(1)-(N) can be communicatively coupled to a respective app subnet 1067(1)-(N) that can be contained in respective container egress VCNs 1068(1)-(N) that can be contained in respective customer tenancies 1070(1)-(N). Respective secondary VNICs 1072(1)-(N) can facilitate communication between the untrusted app subnet(s) 1062 contained in the data plane VCN 1018 and the app subnet contained in the container egress VCNs 1068(1)-(N). Each container egress VCNs 1068(1)-(N) can include a NAT gateway 1038 that can be communicatively coupled to public Internet 1054 (e.g., public Internet 854 of FIG. 8).

The Internet gateway 1034 contained in the control plane VCN 1016 and contained in the data plane VCN 1018 can be communicatively coupled to a metadata management service 1052 (e.g., the metadata management system 852 of FIG. 8) that can be communicatively coupled to public Internet 1054. Public Internet 1054 can be communicatively coupled to the NAT gateway 1038 contained in the control plane VCN 1016 and contained in the data plane VCN 1018. The service gateway 1036 contained in the control plane VCN 1016 and contained in the data plane VCN 1018 can be communicatively coupled to cloud services 1056.

In some embodiments, the data plane VCN 1018 can be integrated with customer tenancies 1070. This integration can be useful or desirable for customers of the IaaS provider in some cases such as a case that may desire support when executing code. The customer may provide code to run that may be destructive, may communicate with other customer resources, or may otherwise cause undesirable effects. In response to this, the IaaS provider may determine whether to run code given to the IaaS provider by the customer.

In some examples, the customer of the IaaS provider may grant temporary network access to the IaaS provider and request a function to be attached to the data plane app tier 1046. Code to run the function may be executed in the VMs 1066(1)-(N), and the code may not be configured to run anywhere else on the data plane VCN 1018. Each VM 1066(1)-(N) may be connected to one customer tenancy 1070. Respective containers 1071(1)-(N) contained in the VMs 1066(1)-(N) may be configured to run the code. In this case, there can be a dual isolation (e.g., the containers 1071(1)-(N) running code, where the containers 1071(1)-(N) may be contained in at least the VM 1066(1)-(N) that are contained in the untrusted app subnet(s) 1062), which may help prevent incorrect or otherwise undesirable code from damaging the network of the IaaS provider or from damaging a network of a different customer. The containers 1071(1)-(N) may be communicatively coupled to the customer tenancy 1070 and may be configured to transmit or receive data from the customer tenancy 1070. The containers 1071(1)-(N) may not be configured to transmit or receive data from any other entity in the data plane VCN 1018. Upon completion of running the code, the IaaS provider may kill or otherwise dispose of the containers 1071(1)-(N).

In some embodiments, the trusted app subnet(s) 1060 may run code that may be owned or operated by the IaaS provider. In this embodiment, the trusted app subnet(s) 1060 may be communicatively coupled to the DB subnet(s) 1030 and be configured to execute CRUD operations in the DB subnet(s) 1030. The untrusted app subnet(s) 1062 may be communicatively coupled to the DB subnet(s) 1030, but in this embodiment, the untrusted app subnet(s) may be configured to execute read operations in the DB subnet(s) 1030. The containers 1071(1)-(N) that can be contained in the VM 1066(1)-(N) of each customer and that may run code from the customer may not be communicatively coupled with the DB subnet(s) 1030.

In other embodiments, the control plane VCN 1016 and the data plane VCN 1018 may not be directly communicatively coupled. In this embodiment, there may be no direct communication between the control plane VCN 1016 and the data plane VCN 1018. However, communication can occur indirectly through at least one method. An LPG 1010 may be established by the IaaS provider that can facilitate communication between the control plane VCN 1016 and the data plane VCN 1018. In another example, the control plane VCN 1016 or the data plane VCN 1018 can make a call to cloud services 1056 via the service gateway 1036. For example, a call to cloud services 1056 from the control plane VCN 1016 can include a request for a service that can communicate with the data plane VCN 1018.

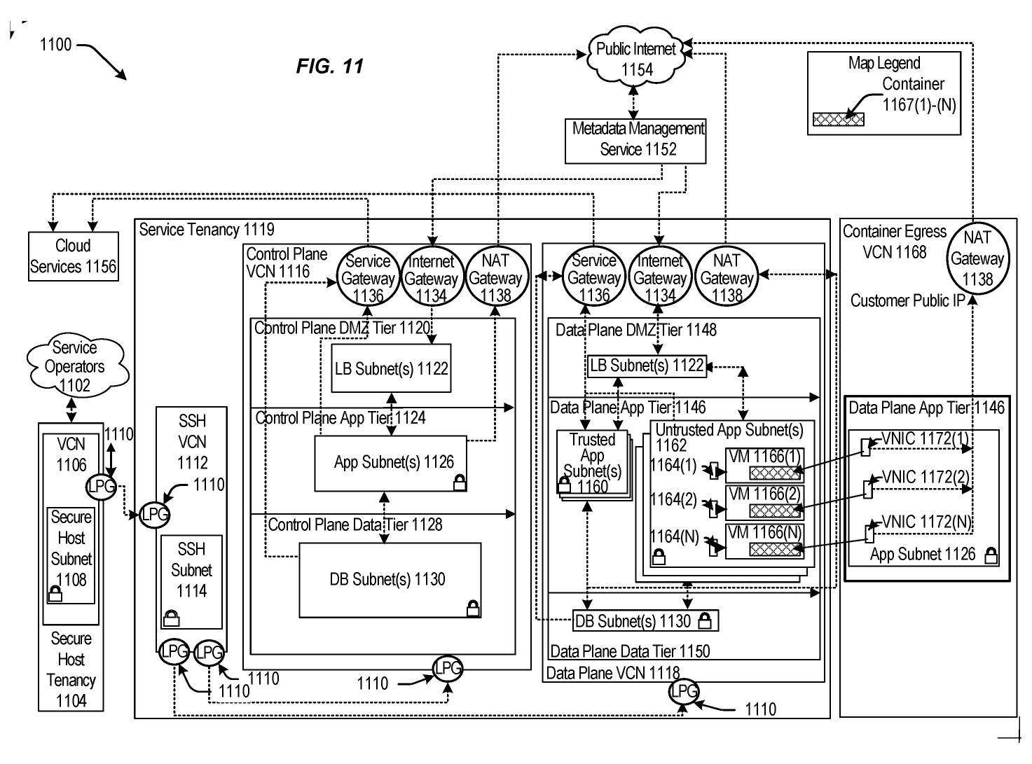

FIG. 11 is a block diagram 1100 illustrating another example pattern of an IaaS architecture, according to at least one embodiment. Service operators 1102 (e.g., service operators 802 of FIG. 8) can be communicatively coupled to a secure host tenancy 1104 (e.g., the secure host tenancy 804 of FIG. 8) that can include a virtual cloud network (VCN) 1106 (e.g., the VCN 806 of FIG. 8) and a secure host subnet 1108 (e.g., the secure host subnet 808 of FIG. 8). The VCN 1106 can include an LPG 1110 (e.g., the LPG 810 of FIG. 8) that can be communicatively coupled to an SSH VCN 1112 (e.g., the SSH VCN 812 of FIG. 8) via an LPG 1110 contained in the SSH VCN 1112. The SSH VCN 1112 can include an SSH subnet 1114 (e.g., the SSH subnet 814 of FIG. 8), and the SSH VCN 1112 can be communicatively coupled to a control plane VCN 1116 (e.g., the control plane VCN 816 of FIG. 8) via an LPG 1110 contained in the control plane VCN 1116 and to a data plane VCN 1118 (e.g., the data plane 818 of FIG. 8) via an LPG 1110 contained in the data plane VCN 1118. The control plane VCN 1116 and the data plane VCN 1118 can be contained in a service tenancy 1119 (e.g., the service tenancy 819 of FIG. 8).

The control plane VCN 1116 can include a control plane DMZ tier 1120 (e.g., the control plane DMZ tier 820 of FIG. 8) that can include LB subnet(s) 1122 (e.g., LB subnet(s) 822 of FIG. 8), a control plane app tier 1124 (e.g., the control plane app tier 824 of FIG. 8) that can include app subnet(s) 1126 (e.g., app subnet(s) 826 of FIG. 8), a control plane data tier 1128 (e.g., the control plane data tier 828 of FIG. 8) that can include DB subnet(s) 1130 (e.g., DB subnet(s) 1030 of FIG. 10). The LB subnet(s) 1122 contained in the control plane DMZ tier 1120 can be communicatively coupled to the app subnet(s) 1126 contained in the control plane app tier 1124 and to an Internet gateway 1134 (e.g., the Internet gateway 834 of FIG. 8) that can be contained in the control plane VCN 1116, and the app subnet(s) 1126 can be communicatively coupled to the DB subnet(s) 1130 contained in the control plane data tier 1128 and to a service gateway 1136 (e.g., the service gateway of FIG. 8) and a network address translation (NAT) gateway 1138 (e.g., the NAT gateway 838 of FIG. 8). The control plane VCN 1116 can include the service gateway 1136 and the NAT gateway 1138.

The data plane VCN 1118 can include a data plane app tier 1146 (e.g., the data plane app tier 846 of FIG. 8), a data plane DMZ tier 1148 (e.g., the data plane DMZ tier 848 of FIG. 8), and a data plane data tier 1150 (e.g., the data plane data tier 850 of FIG. 8). The data plane DMZ tier 1148 can include LB subnet(s) 1122 that can be communicatively coupled to trusted app subnet(s) 1160 (e.g., trusted app subnet(s) 1060 of FIG. 10) and untrusted app subnet(s) 1162 (e.g., untrusted app subnet(s) 1062 of FIG. 10) of the data plane app tier 1146 and the Internet gateway 1134 contained in the data plane VCN 1118. The trusted app subnet(s) 1160 can be communicatively coupled to the service gateway 1136 contained in the data plane VCN 1118, the NAT gateway 1138 contained in the data plane VCN 1118, and DB subnet(s) 1130 contained in the data plane data tier 1150. The untrusted app subnet(s) 1162 can be communicatively coupled to the service gateway 1136 contained in the data plane VCN 1118 and DB subnet(s) 1130 contained in the data plane data tier 1150. The data plane data tier 1150 can include DB subnet(s) 1130 that can be communicatively coupled to the service gateway 1136 contained in the data plane VCN 1118.

The untrusted app subnet(s) 1162 can include primary VNICs 1164(1)-(N) that can be communicatively coupled to tenant virtual machines (VMs) 1166(1)-(N) residing within the untrusted app subnet(s) 1162. Each tenant VM 1166(1)-(N) can run code in a respective container 1167(1)-(N), and be communicatively coupled to an app subnet 1126 that can be contained in a data plane app tier 1146 that can be contained in a container egress VCN 1168. Respective secondary VNICs 1172(1)-(N) can facilitate communication between the untrusted app subnet(s) 1162 contained in the data plane VCN 1118 and the app subnet contained in the container egress VCN 1168. The container egress VCN can include a NAT gateway 1138 that can be communicatively coupled to public Internet 1154 (e.g., public Internet 854 of FIG. 8).

The Internet gateway 1134 contained in the control plane VCN 1116 and contained in the data plane VCN 1118 can be communicatively coupled to a metadata management service 1152 (e.g., the metadata management system 852 of FIG. 8) that can be communicatively coupled to public Internet 1154. Public Internet 1154 can be communicatively coupled to the NAT gateway 1138 contained in the control plane VCN 1116 and contained in the data plane VCN 1118. The service gateway 1136 contained in the control plane VCN 1116 and contained in the data plane VCN 1118 can be communicatively coupled to cloud services 1156.

In some examples, the pattern illustrated by the architecture of block diagram 1100 of FIG. 11 may be considered an exception to the pattern illustrated by the architecture of block diagram 1000 of FIG. 10 and may be desirable for a customer of the IaaS provider if the IaaS provider cannot directly communicate with the customer (e.g., a disconnected region). The respective containers 1167(1)-(N) that are contained in the VMs 1166(1)-(N) for each customer can be accessed in real-time by the customer. The containers 1167(1)-(N) may be configured to make calls to respective secondary VNICs 1172(1)-(N) contained in app subnet(s) 1126 of the data plane app tier 1146 that can be contained in the container egress VCN 1168. The secondary VNICs 1172(1)-(N) can transmit the calls to the NAT gateway 1138 that may transmit the calls to public Internet 1154. In this example, the containers 1167(1)-(N) that can be accessed in real-time by the customer can be isolated from the control plane VCN 1116 and can be isolated from other entities contained in the data plane VCN 1118. The containers 1167(1)-(N) may also be isolated from resources from other customers.

In other examples, the customer can use the containers 1167(1)-(N) to call cloud services 1156. In this example, the customer may run code in the containers 1167(1)-(N) that requests a service from cloud services 1156. The containers 1167(1)-(N) can transmit this request to the secondary VNICs 1172(1)-(N) that can transmit the request to the NAT gateway that can transmit the request to public Internet 1154. Public Internet 1154 can transmit the request to LB subnet(s) 1122 contained in the control plane VCN 1116 via the Internet gateway 1134. In response to determining the request is valid, the LB subnet(s) can transmit the request to app subnet(s) 1126 that can transmit the request to cloud services 1156 via the service gateway 1136.

It should be appreciated that IaaS architectures 800, 900, 1000, 1100 depicted in the figures may have other components than those depicted. Further, the embodiments shown in the figures are only some examples of a cloud infrastructure system that may incorporate an embodiment of the disclosure. In some other embodiments, the IaaS systems may have more or fewer components than shown in the figures, may combine two or more components, or may have a different configuration or arrangement of components.

In certain embodiments, the IaaS systems described herein may include a suite of applications, middleware, and database service offerings that are delivered to a customer in a self-service, subscription-based, elastically scalable, reliable, highly available, and secure manner. An example of such an IaaS system is the Oracle Cloud Infrastructure (OCI) provided by the present assignee.

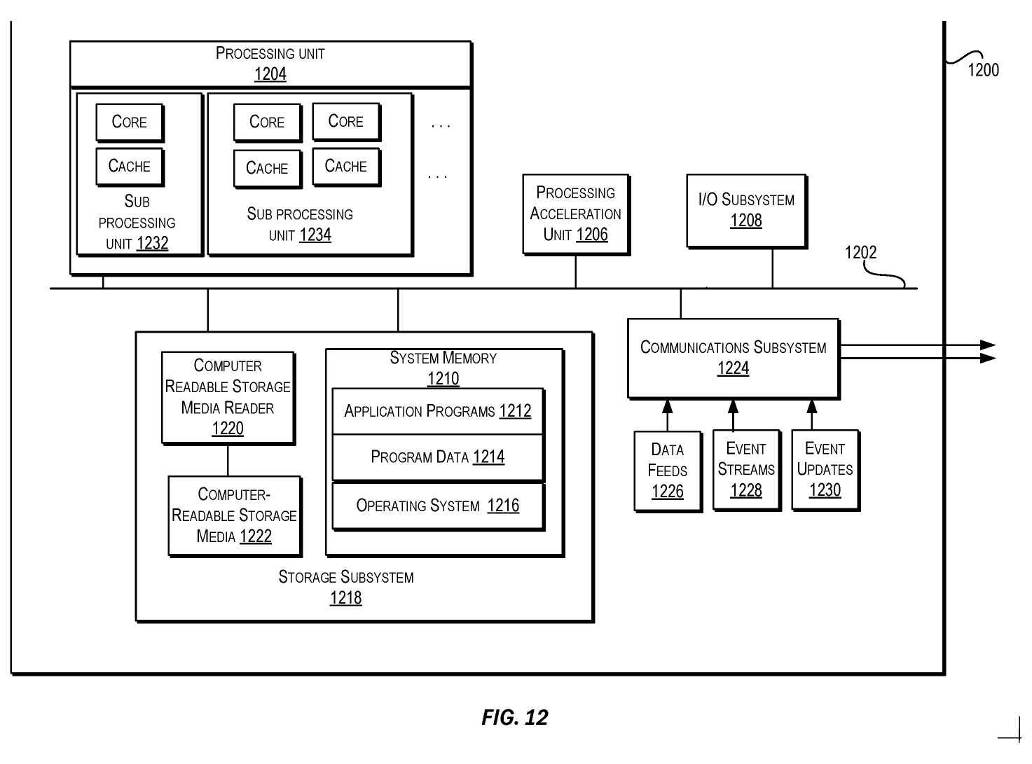

FIG. 12 illustrates an example computer system 1200, in which various embodiments may be implemented. The system 1200 may be used to implement any of the computer systems described above. As shown in the figure, computer system 1200 includes a processing unit 1204 that communicates with a number of peripheral subsystems via a bus subsystem 1202. These peripheral subsystems may include a processing acceleration unit 1206, an I/O subsystem 1208, a storage subsystem 1218 and a communications subsystem 1224. Storage subsystem 1218 includes tangible computer-readable storage media 1222 and a system memory 1210.

Bus subsystem 1202 provides a mechanism for letting the various components and subsystems of computer system 1200 communicate with each other as intended. Although bus subsystem 1202 is shown schematically as a single bus, alternative embodiments of the bus subsystem may utilize multiple buses. Bus subsystem 1202 may be any of several types of bus structures including a memory bus or memory controller, a peripheral bus, and a local bus using any of a variety of bus architectures. For example, such architectures may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P1386.1 standard.

Processing unit 1204, which can be implemented as one or more integrated circuits (e.g., a conventional microprocessor or microcontroller), controls the operation of computer system 1200. One or more processors may be included in processing unit 1204. These processors may include single core or multicore processors. In certain embodiments, processing unit 1204 may be implemented as one or more independent processing units 1232 and/or 1234 with single or multicore processors included in each processing unit. In other embodiments, processing unit 1204 may also be implemented as a quad-core processing unit formed by integrating two dual-core processors into a single chip.

In various embodiments, processing unit 1204 can execute a variety of programs in response to program code and can maintain multiple concurrently executing programs or processes. At any given time, some or all of the program code to be executed can be resident in processor(s) 1204 and/or in storage subsystem 1218. Through suitable programming, processor(s) 1204 can provide various functionalities described above. Computer system 1200 may additionally include a processing acceleration unit 1206, which can include a digital signal processor (DSP), a special-purpose processor, and/or the like.

I/O subsystem 1208 may include user interface input devices and user interface output devices. User interface input devices may include a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may include, for example, motion sensing and/or gesture recognition devices such as the Microsoft Kinect® motion sensor that enables users to control and interact with an input device, such as the Microsoft Xbox® 360 game controller, through a natural user interface using gestures and spoken commands. User interface input devices may also include eye gesture recognition devices such as the Google Glass® blink detector that detects eye activity (e.g., ‘blinking’ while taking pictures and/or making a menu selection) from users and transforms the eye gestures as input into an input device (e.g., Google Glass®). Additionally, user interface input devices may include voice recognition sensing devices that enable users to interact with voice recognition systems (e.g., Siri® navigator), through voice commands.

User interface input devices may also include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, barcode reader 3D scanners, 3D printers, laser rangefinders, and eye gaze tracking devices. Additionally, user interface input devices may include, for example, medical imaging input devices such as computed tomography, magnetic resonance imaging, position emission tomography, medical ultrasonography devices. User interface input devices may also include, for example, audio input devices such as MIDI keyboards, digital musical instruments and the like.

User interface output devices may include a display subsystem, indicator lights, or non-visual displays such as audio output devices, etc. The display subsystem may be a cathode ray tube (CRT), a flat-panel device, such as that using a liquid crystal display (LCD) or plasma display, a projection device, a touch screen, and the like. In general, use of the term "output device" is intended to include all possible types of devices and mechanisms for outputting information from computer system 1200 to a user or other computer. For example, user interface output devices may include, without limitation, a variety of display devices that visually convey text, graphics and audio/video information such as monitors, printers, speakers, headphones, automotive navigation systems, plotters, voice output devices, and modems.

Computer system 1200 may comprise a storage subsystem 1218 that provides a tangible non-transitory computer-readable storage medium for storing software and data constructs that provide the functionality of the embodiments described in this disclosure. The software can include programs, code modules, instructions, scripts, etc., that when executed by one or more cores or processors of processing unit 1204 provide the functionality described above. Storage subsystem 1218 may also provide a repository for storing data used in accordance with the present disclosure.

As depicted in the example in FIG. 12, storage subsystem 1218 can include various components including a system memory 1210, computer-readable storage media 1222, and a computer readable storage media reader 1220. System memory 1210 may store program instructions that are loadable and executable by processing unit 1204. System memory 1210 may also store data that is used during the execution of the instructions and/or data that is generated during the execution of the program instructions. Various different kinds of programs may be loaded into system memory 1210 including but not limited to client applications, Web browsers, mid-tier applications, relational database management systems (RDBMS), virtual machines, containers, etc.

System memory 1210 may also store an operating system 1216. Examples of operating system 1216 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems such as iOS, Windows® Phone, Android® OS, BlackBerry® OS, and Palm® OS operating systems. In certain implementations where computer system 1200 executes one or more virtual machines, the virtual machines along with their guest operating systems (GOSs) may be loaded into system memory 1210 and executed by one or more processors or cores of processing unit 1204.

System memory 1210 can come in different configurations depending upon the type of computer system 1200. For example, system memory 1210 may be volatile memory (such as random access memory (RAM)) and/or non-volatile memory (such as read-only memory (ROM), flash memory, etc.) Different types of RAM configurations may be provided including a static random access memory (SRAM), a dynamic random access memory (DRAM), and others. In some implementations, system memory 1210 may include a basic input/output system (BIOS) containing basic routines that help to transfer information between elements within computer system 1200, such as during start-up.

Computer-readable storage media 1222 may represent remote, local, fixed, and/or removable storage devices plus storage media for temporarily and/or more permanently containing, storing, computer-readable information for use by computer system 1200 including instructions executable by processing unit 1204 of computer system 1200.

Computer-readable storage media 1222 can include any appropriate media known or used in the art, including storage media and communication media, such as but not limited to, volatile and non-volatile, removable and non-removable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media.

By way of example, computer-readable storage media 1222 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media. Computer-readable storage media 1222 may include, but is not limited to, Zip® drives, flash memory cards, universal serial bus (USB) flash drives, secure digital (SD) cards, DVD disks, digital video tape, and the like. Computer-readable storage media 1222 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magnetoresistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for computer system 1200.

Machine-readable instructions executable by one or more processors or cores of processing unit 1204 may be stored on a non-transitory computer-readable storage medium. A non-transitory computer-readable storage medium can include physically tangible memory or storage devices that include volatile memory storage devices and/or non-volatile storage devices. Examples of non-transitory computer-readable storage medium include magnetic storage media (e.g., disk or tapes), optical storage media (e.g., DVDs, CDs), various types of RAM, ROM, or flash memory, hard drives, floppy drives, detachable memory drives (e.g., USB drives), or other type of storage device.

Communications subsystem 1224 provides an interface to other computer systems and networks. Communications subsystem 1224 serves as an interface for receiving data from and transmitting data to other systems from computer system 1200. For example, communications subsystem 1224 may enable computer system 1200 to connect to one or more devices via the Internet. In some embodiments communications subsystem 1224 can include radio frequency (RF) transceiver components for accessing wireless voice and/or data networks (e.g., using cellular telephone technology, advanced data network technology, such as 3G, 4G or EDGE (enhanced data rates for global evolution), WiFi (IEEE 802.11 family standards, or other mobile communication technologies, or any combination thereof)), global positioning system (GPS) receiver components, and/or other components. In some embodiments communications subsystem 1224 can provide wired network connectivity (e.g., Ethernet) in addition to or instead of a wireless interface.

In some embodiments, communications subsystem 1224 may also receive input communication in the form of structured and/or unstructured data feeds 1226, event streams 1228, event updates 1230, and the like on behalf of one or more users who may use computer system 1200.

By way of example, communications subsystem 1224 may be configured to receive data feeds 1226 in real-time from users of social networks and/or other communication services such as Twitter® feeds, Facebook® updates, web feeds such as Rich Site Summary (RSS) feeds, and/or real-time updates from one or more third party information sources.

Additionally, communications subsystem 1224 may also be configured to receive data in the form of continuous data streams, which may include event streams 1228 of real-time events and/or event updates 1230, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 1224 may also be configured to output the structured and/or unstructured data feeds 1226, event streams 1228, event updates 1230, and the like to one or more databases that may be in communication with one or more streaming data source computers coupled to computer system 1200.

Computer system 1200 can be one of various types, including a handheld portable device (e.g., an iPhone® cellular phone, an iPad® computing tablet, a PDA), a wearable device (e.g., a Google Glass® head mounted display), a PC, a workstation, a mainframe, a kiosk, a server rack, or any other data processing system.

Due to the ever-changing nature of computers and networks, the description of computer system 1200 depicted in the figure is intended only as a specific example. Many other configurations having more or fewer components than the system depicted in the figure are possible. For example, customized hardware might also be used and/or particular elements might be implemented in hardware, firmware, software (including applets), or a combination. Further, connection to other computing devices, such as network input/output devices, may be employed. Based on the disclosure and teachings provided herein, a person of ordinary skill in the art will appreciate other ways and/or methods to implement the various embodiments.

Although specific embodiments have been described, various modifications, alterations, alternative constructions, and equivalents are also encompassed within the scope of the disclosure. Embodiments are not restricted to operation within certain specific data processing environments, but are free to operate within a plurality of data processing environments. Additionally, although embodiments have been described using a particular series of transactions and steps, it should be apparent to those skilled in the art that the scope of the present disclosure is not limited to the described series of transactions and steps. Various features and aspects of the above-described embodiments may be used individually or jointly.

Further, while embodiments have been described using a particular combination of hardware and software, it should be recognized that other combinations of hardware and software are also within the scope of the present disclosure. Embodiments may be implemented only in hardware, or only in software, or using combinations thereof. The various processes described herein can be implemented on the same processor or different processors in any combination. Accordingly, where components or services are described as being configured to perform certain operations, such configuration can be accomplished, e.g., by designing electronic circuits to perform the operation, by programming programmable electronic circuits (such as microprocessors) to perform the operation, or any combination thereof. Processes can communicate using a variety of techniques including but not limited to conventional techniques for inter process communication, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific disclosure embodiments have been described, these are not intended to be limiting. Various modifications and equivalents are within the scope of the following claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Preferred embodiments of this disclosure are described herein, including the best mode known for carrying out the disclosure. Variations of those preferred embodiments may become apparent to those of ordinary skill in the art upon reading the foregoing description. Those of ordinary skill should be able to employ such variations as appropriate and the disclosure may be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein.

All references, including publications, patent applications, and patents, cited herein are hereby incorporated by reference to the same extent as if each reference were individually and specifically indicated to be incorporated by reference and were set forth in its entirety herein.

In the foregoing specification, aspects of the disclosure are described with reference to specific embodiments thereof, but those skilled in the art will recognize that the disclosure is not limited thereto. Various features and aspects of the above-described disclosure may be used individually or jointly. Further, embodiments can be utilized in any number of environments and applications beyond those described herein without departing from the broader spirit and scope of the specification. The specification and drawings are, accordingly, to be regarded as illustrative rather than restrictive.

What is claimed is:

1. A computer-implemented method comprising:
receiving a request to provide a summary of patient-specific information regarding a medication for a particular patient;
retrieving data relevant to the request from a plurality of sources, wherein the retrieved data includes at least structured and semi-structured content;
harmonizing the retrieved data for at least data structure and semantic alignment to generate harmonized data;
filtering the harmonized data to extract fields in the harmonized data relevant to the request;
generating an input for a generative machine learning model, the input comprising a prompt generated based on the request;
generating, by the generative machine learning model, a query result associated with the input, the query result comprising a subset of the filtered, harmonized data;
formatting the query result into an output, the output comprising the summary of patient-specific information regarding the medication; and
providing the output to a client system.

2. The computer-implemented method of claim 1, wherein harmonizing the data includes transforming the retrieved data into a data format digestible by the generative machine learning model.

3. The computer-implemented method of claim 2, wherein
the data includes a prescription history for the patient and guidance specific to the medication, and
retrieving the data includes
accessing the prescription history from an electronic health record (EHR) and
obtaining the guidance specific to the medication from a medication database.

4. The computer-implemented method of claim 3, wherein
providing the prompt to the generative machine learning model includes
instructing the generative machine learning model to analyze the prescription history and the guidance specific to the medication for contraindication for the medication in view of the prescription history.

5. The computer-implemented method of claim 3, wherein
the data further includes additional information including at least one of the patient’s health history, the patient’s recent laboratory results, the patient’s vitals, and one or more clinical notes from the patient’s recent visits with a clinician making the request, and
retrieving the data further includes accessing the additional information from the EHR.

6. The computer-implemented method of claim 3, wherein
the data further includes insurance coverage information, and
retrieving the data relevant to the summary further includes
accessing the insurance coverage information from an insurance provider database.

7. The computer-implemented method of claim 6, wherein
providing the prompt to the machine learning generative model includes instructing the machine learning generative model to review the insurance coverage information for the medication.

8. The computer-implemented method of claim 1, wherein providing the output to the client system includes
presenting, at the client system, a medication summary report including at least one of a narrative summary section and a structured section,
the narrative summary including a first set of selections from the subset of the filtered, harmonized data arranged into natural language text, and
the structured section including a second set of selections from the subset of the filtered, harmonized data used to populate a plurality of preset windows within the medication summary report.

9. The computer-implemented method of claim 8, wherein at least one of the narrative summary and the structured section includes one of a Uniform Resource Locator (URL) and a hovering window to enable display of additional information regarding a specific portion of the summary of patient-specific information.

10. A system comprising:
one or more processors; and
one or more computer-readable media storing instructions which, when executed by the one or more processors, cause the system to perform operations comprising:
receiving a request to provide a summary of patient-specific information regarding a medication for a particular patient;
retrieving data relevant to the request from a plurality of sources, wherein the retrieved data includes at least structured and semi-structured content;
harmonizing the retrieved data for at least data structure and semantic alignment to generate harmonized data;
filtering the harmonized data to extract fields in the harmonized data relevant to the request;
generating an input for a generative machine learning model, the input comprising a prompt generated based on the request;
generating, by the generative machine learning model, a query result associated with the input, the query result comprising a subset of the filtered, harmonized data;
formatting the query result into an output, the output comprising the summary of patient-specific information regarding the medication; and
providing the output to a client system.

11. The system of claim 10, wherein harmonizing the data includes transforming the data into a data format digestible by the generative machine learning model.

12. The system of claim 11, wherein
the data includes a prescription history for the patient and guidance specific to the medication, and
retrieving the data includes
accessing the prescription history from an electronic health record (EHR) and
obtaining the guidance specific to the medication from a medication database.

13. The system of claim 12, wherein
providing the prompt to the generative machine learning model includes
instructing the generative machine learning model to analyze the prescription history and the guidance specific to the medication for contraindication for the medication in view of the prescription history.

14. The system of claim 12, wherein
the data further includes additional information including at least one of the patient’s health history, the patient’s recent laboratory results, the patient’s vitals, and one or more clinical notes from the patient’s recent visits with a clinician making the request, and
retrieving the data further includes accessing the additional information from the EHR.

15. The system of claim 12, wherein
the data further includes insurance coverage information, and
retrieving the data relevant to the summary further includes
accessing the insurance coverage information from an insurance provider database.

16. The system of claim 15, wherein
providing the prompt to the generative machine learning model includes instructing the generative machine learning model to review the insurance coverage information for the medication.

17. The system of claim 10, wherein providing the output to the client system includes
presenting, at the client system, a medication summary report including at least one of a narrative summary section and a structured section,
the narrative summary including a first set of selections from the subset of the filtered, harmonized data arranged into natural language text, and
the structured section including a second set of selections from the subset of the filtered, harmonized data used to populate a plurality of preset windows within the medication summary report.

18. The system of claim 17, wherein at least one of the narrative summary and the structured section includes one of a Uniform Resource Locator (URL) and a hovering window to enable display of additional information regarding a specific portion of the summary of patient-specific information.

19. One or more non-transitory computer-readable media storing instructions which, when executed by one or more processors, cause the one or more processors to perform operations comprising:
receiving a request to provide a summary of patient-specific information regarding a medication for a particular patient;
retrieving data relevant to the request from a plurality of sources, wherein the retrieved data includes at least structured and semi-structured content;
harmonizing the retrieved data for at least data structure and semantic alignment to generate harmonized data;
filtering the harmonized data to extract fields in the harmonized data relevant to the request;
generating an input for a generative machine learning model, the input comprising a prompt generated based on the request;
generating, by the generative machine learning model, a query result associated with the input, the query result comprising a subset of the filtered, harmonized data;
formatting the query result into an output, the output comprising the summary of patient-specific information regarding the medication; and
providing the output to a client system.

20. The one or more non-transitory computer-readable media of claim 19, wherein harmonizing the data includes transforming the data into a data format digestible by the generative machine learning model.
